(19) United States
(12) Patent Application Publication
(10) Pub. No.: US 2009/0291146 A1
(43) Pub. Date: Nov. 26, 2009

(54) PROCESS FOR MANUFACTURING LACTOSE

(75) Inventors: Trevor Charles Roche, Hertfordshire (GB); Marian Wladyslaw Wood-Kaczmar, Hertfordshire (GB)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(73) Assignee: GLAXO GROUP LIMITED

(21) Appl. No.: 12/375,716

(22) PCT Filed: Aug. 8, 2007
(86) PCT No.: PCT/US07/17647

§ 371 Date: Jan. 30, 2009

Related U.S. Application Data
(60) Provisional application No. 60/821,872, filed on Aug. 9, 2006.

(51) Int. Cl. A61K 9/14; C13K 5/00; A61K 47/26; A61K 31/135; A61K 31/566

(52) U.S. Cl. 424/499; 536/123.13; 514/777; 514/651; 514/653; 514/180

(57) ABSTRACT

A process for producing lactose particles comprises combining a predetermined quantity of lactose seed particles to a first aqueous solution comprising a plurality of lactose particles to form a second solution, wherein the predetermined quantity of lactose seed particles is present in a well dispersed suspension and the first aqueous solution is saturated with said plurality of lactose particles; and subjecting the second solution to conditions sufficient to induce crystallization of the lactose seed particles to form a second plurality of lactose particles having a median particle size of about 25 microns to about 100 microns.

### FIELD OF INVENTION

The invention generally relates to processes for producing lactose particles.

### BACKGROUND OF THE INVENTION

In the field of inhalation therapy, it is generally desirable to employ therapeutic molecules having a particle size (i.e., diameter) in the range of 1 to 5 μm. Carrier molecules or excipients, such as lactose, for inhaled therapeutic preparations often exhibit a significantly larger diameter (e.g., 100 to 150 μm) so that they typically do not penetrate into the upper respiratory tract to the same degree as the active ingredient. However, in many instances, it is desired to use a smaller particle size for the lactose or a lactose blend having a defined ratio of coarse and fine lactose.

The lactose particle size and distribution may also, in many instances, significantly influence pharmaceutical and biological properties, such as, for example, flow properties, cohesiveness, or bioavailability.

It is believed that one particular drawback associated with conventional means of producing pharmaceutical grade lactose relates to undesirable variations in particle size, morphology and distribution. Such production methods may be particularly problematic in that they often lead to excessive and undesirable variations in the fine particle mass (“FPMass”) of pharmaceutical formulations employing such lactose. FPMass is the weight of medicament within a given dose that reaches the desired size airways to be effective.

It would be desirable to employ a process capable of producing lactose having a more consistent particle size distribution.

### SUMMARY OF THE INVENTION

In one aspect, the invention provides a process for producing lactose particles comprising combining a predetermined quantity of lactose seed particles to a first aqueous solution comprising a plurality of lactose particles to form a second solution, wherein the predetermined quantity of lactose seed particles is present in a de-agglomerated suspension and the first aqueous solution is supersaturated with said plurality of lactose particles; subjecting the second solution to conditions sufficient to induce crystallization of the lactose seed particles to form a second plurality of lactose particles having a median particle size of about 25 microns to about 100 microns.

These and other aspects are provided by the present invention.

### BRIEF DESCRIPTION OF THE DRAWINGS

FIG. 1 is an SEM photograph of seed lactose used in Examples 3-47 of the present invention.

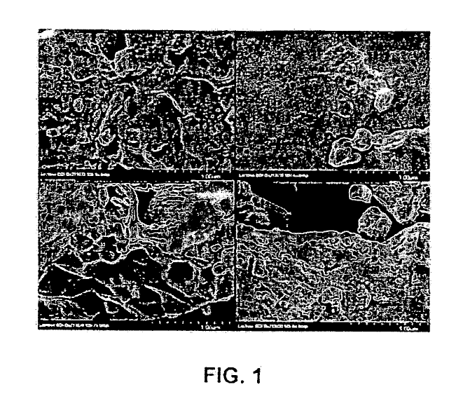

FIG. 2 is an SEM photograph of seed lactose used in Examples 48, 51-57 and 76 of the present invention.

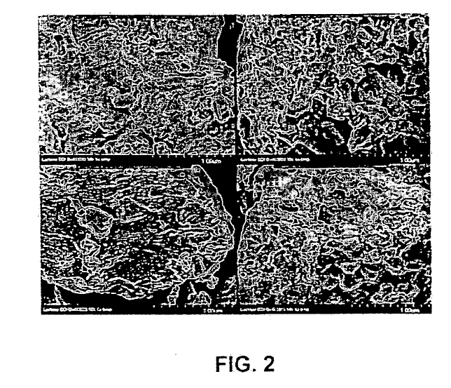

FIG. 3 is an SEM photograph of seed lactose used in Example 49 of the present invention.

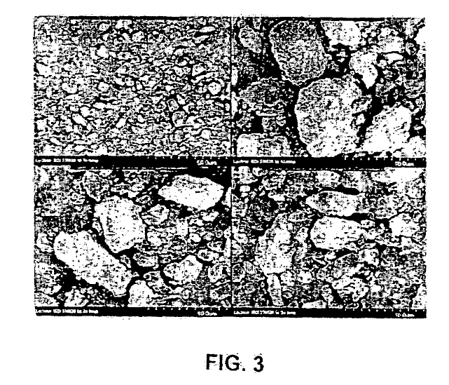

FIG. 4 is a graph illustrating FPM for various formulations

### DETAILED DESCRIPTION OF THE INVENTION

The invention will now be described with respect to the embodiments set forth herein including those alluded to in the drawings. It should be appreciated that these embodiments are set forth to illustrate the invention, and that the invention is not limited to these embodiments. Such embodiments may or may not be practiced mutually exclusive of each other.

All publications, patents, and patent applications cited herein, whether supra or infra, are hereby incorporated herein by reference to their entirety to the same extent as if each publication, patent, or patent application was specifically and individually indicated to be incorporated by reference.

It must be noted that, as used in the specification and appended claims, the singular forms “a”, “an”, “the” and “one” include plural referents unless the content clearly dictates otherwise.

The term “X50” as used herein refers to the median diameter (μm) as measured on a volume basis by a laser diffraction particle sizing system. Particle size analysis performed on either Sympatec HELOS system H0933 or Malvern Mastersizer 2000. 50% by volume of the particles are smaller than this diameter and 50% are larger.

In accordance with the present invention, the term “lactose” as used herein is to be broadly construed. As an example, lactose is intended to encompass physical, crystalline, amorphous and polymorphic forms of lactose, including, but not limited to, the stereoisomers α-lactose monohydrate and β-anhydrous lactose, as well as α-anhydrous lactose. Combinations of the above may be used. Lactose (i.e., milk sugar) is preferably obtained from cheese-whey, which can be manufactured in different forms depending on the process employed. In one embodiment, the plurality of lactose particles comprise α-lactose monohydrate. In one embodiment, the plurality of lactose particles consist essentially of α-lactose monohydrate. In one embodiment, the plurality of lactose particles consist of α-lactose monohydrate. In one embodiment, the α-lactose monohydrate may have an anomeric purity of at least ninety-six (96) percent. The term “coarse lactose” as used herein is to be interpreted as lactose with a median diameter (“X50”) of approximately 25 to 125 micrometers. As used herein, the term “particle” is to be broadly interpreted to encompass those of various shapes, sizes, and/or textures which can include those that may have varying degrees of irregularities, and/or disuniformities, or which my possess regular and/or uniform properties. As used herein “seed particles” is to be broadly construed to encompass lactose particles, as individually described herein, employed to initiate crystallization.

The lactose employed (i.e., “seed particles”) in the process of the invention may have various size distributions. As an example, the X50 of the seed particles may have a size ranging from 1, 2 or 3 microns to about 5, 6 or 7 microns as a realistic range of seeds that can be used for this invention.

The seed particles that comprise a plurality of lactose seed particles may be in various solutions, any of which may be referred to as a seed suspension (“seed suspension”). For example, in one embodiment, the seed suspension is a slurry of lactose seed particles in a water miscible nonsolvent, any of which may be referred to as a seed slurry (“seed slurry”). The term “miscible” as used herein is to be broadly construed to encompass both partially miscible and totally miscible solvents. The term “totally miscible” as used herein is defined as capable of mixing in any ratio without a separation of phases. The term “partially miscible” as used herein is defined as not capable of mixing in all ratios without a separation of phases. In various embodiments, the water miscible nonsolvent may be selected from acetone, methanol, ethanol, tetrahydrofuran, iso-propanol and n-propanol or mixtures thereof. In one embodiment, the water miscible nonsolvent is acetone.

The term “de-agglomerated suspension” as used herein refers to a seed suspension which is shaken, sonicated or otherwise manipulated by one skilled in the art to ensure dispersion of the seed particles in the solution. For example, in one embodiment, the seed suspension may be shaken by hand for 1-3 minutes. In another embodiment, the seed suspension may be sonicated for 15 seconds.

In one embodiment of the invention, a predetermined quantity of lactose Seed particles may be determined according to the equation: m_{Seed}=d³_{Seed}/d³_{product}×m_{product}×c, wherein m_{Seed} represents the mass of the lactose Seed particles; d_{Seed} represents the X50 of the lactose seed particles; d_{product} represents the X50 of the plurality of lactose particles formed by said process; m_{product} represents the mass of the plurality of lactose particles formed by said process; and c represents a seed constant determined by experimental measurement.

In one embodiment of the invention, the seed suspension may be added to a first aqueous solution prior to subjecting to conditions sufficient to cause crystallization to occur on the seed particles. In one embodiment of the invention, the first aqueous solution comprises a plurality of lactose particles. In one embodiment, the first aqueous solution may be a supersaturated lactose solution. As used herein, the term “supersaturated” refers to a condition in which the solvent is holding more solute than is stable at a given temperature. Supersaturation may be defined as the excess concentration of solute over the saturation concentration at a given temperature.

In one embodiment of the invention, the first aqueous solution comprises a base. For example, the base may be NaOH, KOH, LiOH, or NaHCO₃. For example, in one embodiment, the first aqueous solution may contain 0.5 M NaOH. The 0.5 M NaOH may be 1.0, 2.0 or 3.0% solution volume of the first aqueous solution prior to the addition of seed material.

In one embodiment of the invention, the base may be added to the first aqueous solution prior to the addition of the plurality of the lactose seed particles and prior to subjecting the second solution, comprising a seed suspension and the first aqueous solution, to conditions sufficient to cause crystallization. For example, the base may be NaOH, KOH, LiOH, or NaHCO₃. For example, in one embodiment, the base may be 0.5 M NaOH.

In one embodiment, the pH of the second solution may be from about 4 to about 9. In another embodiment, the pH of the second solution may be from about 5 to about 8. In another embodiment, the pH of the second solution may be from about 5 to about 9. In another embodiment, the pH of the second solution may be from about 3 to about 7.

In one embodiment of the invention, the temperature of the first aqueous solution ranges from about 60° C. to about 40° C. In another embodiment of the invention, the temperature of the first aqueous solution is about 50° C.

This invention provides a process for forming crystalline lactose having a specified median diameter. The process comprises subjecting a solution comprising a plurality of lactose particles to conditions sufficient to cause crystallization to occur on lactose seed particles such that a second plurality of lactose particles are formed therefrom. In one embodiment of the invention, the second plurality of lactose particles has a median size of about 50 microns. In one embodiment, the temperature of the second solution having a plurality of lactose particles with a median size of about 50 microns is from about 30° C. to about 0° C. prior.

The step of subjecting a solution comprising a plurality of nanosized lactose particles to conditions sufficient to cause crystallization may occur under various conditions. For example, in one embodiment, such a step may occur such that the solution is linearly cooled at a rate ranging from a lower end of about −0.1, −0.2, −0.3, −0.4, −0.5° C./min to a higher end of about −1, −1.5, −2, −2.5 and −3° C./min. In another embodiment, such a step may occur such that the solution is cooled at a rate of −0.6° C./min. In a third embodiment, such a step may occur such that the solution is cooled by an inverse cooling profile. In one example, not intended to be bound theory, the inverse cooling may follow an inverse cooling curve described by the equation T(t)=Tᵢ−(Tᵢ−T_{f})(t/t_{f})³, where T(t)=temperature at time t, Tᵢ=initial temperature, T_{f}=final temperature and t_{f}=batch time. In a fourth embodiment, such a step may occur such that the solution is step cooled. The term “step cooled” as used herein is defined as a cooling profile in which the solution is slowly cooled at first then cooled more rapidly as crystallization proceeds. The cooling profile may be approximated by a series of linear cooling profiles of gradually increasing cooling rate (eg any curve may be approximated as a series of interconnected straight lines). For example, a seeded solution may be cooled (e.g., step cooled) from 50° C. to 35° C. at −0.21° C./min followed by cooling at −0.57° C./min till 20° C.

The processes of the invention may include further optional features. For example, the resulting crystallized lactose particles (“lactose slurry”) may be optionally subjected to isolation procedures. In one embodiment, the isolation procedures may employ an anti-solvent. The anti-solvent may be selected from the group consisting of acetone, methanol, ethanol, iso-propanol, n-propanol, and tetrahydrofuran and mixtures thereof. In one embodiment, the anti-solvent may be ethanol. The isolated crystallized lactose particles may be optionally subjected to drying procedures. In one embodiment, the crystallized lactose particles may be filtered followed by washing with one (1) excess volume of 20% ethanol/water followed by washing with one (1) excess volume of 40% ethanol/water. The lactose may then be pulled dry for one (1) hour followed by drying in vacuo at 40° C. overnight. In a second embodiment, the lactose slurry may be filtered followed by washing with one (1) excess volume of 40% acetone/water solution. The lactose may then be pulled dry for 0.5 hour followed by drying in vacuo at 40° C. overnight. In an additional embodiment, the lactose slurry may be filtered followed by washing with one (1) excess volume of 40% acetone/water solution followed by washing with one excess volume of acetone. The lactose may then be air dried followed by drying in vacuo at 40° C. overnight. In a third embodiment, the crystallized lactose particles may be dried by techniques including, without limitation, agitated pan drying under vacuum, FIMA fluidized bed drying and drying in a Bolz agitated conical dryer (using an auger to agitate the solid). In addition to the above, it is appreciated that other conditions known in the art may be employed.

In conjunction with the process of the invention, other procedures known in the art can be employed which are often associated with crystallization processes. Examples of such procedures include, without limitation, cleaning and sanitization, vessel pre-wash, and inter-batch cleaning. Many structural configurations may be used. For example, the process of the invention may occur in a commercial vessel. In one embodiment, for example, the process may occur in a De Dietrich Process Systems vessel, 1600 litre capacity (De Dietrich Process Systems, Inc., Union, N.J.) or other standard processing vessels such as Pfaudler-Balfour (Leven, Scotland).

The dried crystallized lactose particles produced in accordance with this invention comprise a plurality of lactose particles having a specified median diameter. The dried crystallized lactose particles may have a X50 ranging from a lower end of about 20, 25, 35, 45, or 55 μm to higher end of about 75, 100, 125, or 150 μm. In one embodiment, one range of median diameters would be about 45 μm to about 75 μm. In another embodiment, a range of median diameters would be about 45 μm to about 100 μm. In a third embodiment, a range of median diameters would be about 35 μm to about 125 μm. In a fourth embodiment, a range of median diameters would be about 20 μm to about 150 μm.

The dried crystallized lactose particles produced in accordance with the described invention may be further combined with an additional plurality of lactose particles having a X50 from a lower end of about 4, 5, 6 or 7 μm to a higher end of about 10, 15 or 20 μm (said additional plurality of lactose particles may be referred to as “fine lactose particles”), producing a blend of lactose particles.

In one embodiment, the crystallized lactose particles produced in accordance with the invention may be combined with at least one medicament to form a pharmaceutical formulation.

In one embodiment, a blend of lactose particles comprising dried crystallized lactose particles produced in accordance with the described invention and an additional plurality of lactose particles having a X50 from a lower end of about 4, 5, 6 or 7 μm to a higher end of about 10, 15 or 20 μm may be combined with at least one medicament to form a pharmaceutical formulation.

In other aspects, the invention may encompass pharmaceutical formulations formed by the processes, as well as inhalation devices including such formulations. For example, the pharmaceutical formulation may be a dry powder pharmaceutical formulation suitable for inhalation. Medicaments, for the purposes of the invention, include a variety of pharmaceutically active ingredients, such as, for example, those which are useful in inhalation therapy. In general, the term “medicament” is to be broadly construed and include, without limitation, actives, drugs and bioactive agents, as well as biopharmaceuticals. Various embodiments may include medicament present in micronized form. Appropriate medicaments may thus be selected from, for example, analgesics, (e.g., codeine, dihydromorphine, ergotamine, fentanyl or morphine); anginal preparations, (e.g., diltiazem); anti-allergics, (e.g., cromoglicate, ketotifen or nedocromil); antiinfectives (e.g., cephalosporins, penicillins, streptomycin, sulphonamides, tetracyclines and pentamidine); antihistamines, (e.g., methapyrilene); anti-inflammatories, (e.g., anti-inflammatory steroids, beclomethasone (e.g. beclomethasone dipropionate), fluticasone (e.g. fluticasone propionate), flunisolide, budesonide, rofleponide, mometasone (e.g. mometasone furoate), ciclesonide, triamcinolone (e.g. triamcinolon acetonide), 6α,9α-difluoro-11β-hydroxy-16α-methyl-3-oxo-17α-propionyloxy-androsta-1,4-diene-17β-carbothioic acid S-(2-oxo-tetrahydro-furan-3-yl)ester), (6α,11β,16α,17α)-6,9-difluoro-17-{[(fluoromethyl)thio]carbonyl}-11-hydroxy-16-methyl-3-oxoandrosta-1,4-dien-17-yl 2-furoate, and (6α,11β,16α,17α)-6,9-difluoro-17-{[(fluoromethyl)thio]carbonyl}-11-hydroxy-16-methyl-3-oxoandrosta-1,4-dien-17-yl 4-methyl-1,3-thiazole-5-carboxylate); antitussives, (e.g., noscapine); bronchodilators, (e.g., albuterol (e.g. as sulphate), salbutamol (e.g. as the free base or the sulphate salt), salmeterol (e.g. as xinafoate), ephedrine, adrenaline, fenoterol (e.g as hydrobromide), bitolterol, formoterol (e.g., as fumarate), isoprenaline, metaproterenol, phenylephrine, phenylpropanolamine, pirbuterol (e.g., as acetate), reproterol (e.g., as hydrochloride), rimiterol, terbutaline (e.g., as sulphate), isoetharine, tulobuterol, 4-hydroxy-7-[2-[[2-[[3-(2-(henylethoxy)propyl]sulfonyl]ethyl]-amino]ethyl-2(3H)-benzothiazolone), 3-(4-{[6-({(2R)-2-hydroxy-2-[4-hydroxy-3-(hydroxymethyl)phenyl]ethyl}amino)hexyl]oxy}butyl)benzenesulfonamide, 3-(3-{[7-({(2R)-2-hydroxy-2-[4-hydroxy-3-(hydroxymethyl)phenyl]ethyl}amino)heptyl]oxy}propyl)benzenesulfonamide, 4-{(1R)-2-[(6-{2-[(2,6-dichlorobenzyl)oxy]ethoxy}hexyl)amino]-1-hydroxyethyl}-2-(hydroxymethyl)phenol, 2-hydroxy-5-((1R)-1-hydroxy-2-{[2-(4-{[(2R)-2-hydroxy-2-phenylethyl]amino}phenyl)ethyl]amino}ethyl)phenylformamide, and 8-hydroxy-5-{(1R)-1-hydroxy-2-[(2-{4-[(6-methoxy-1,1′-biphenyl-3-yl)amino]phenyl}ethyl)amino]ethyl}quinolin-2(1H)-one); diuretics, (e.g., amiloride); anticholinergics, (e.g., ipatropium (e.g., as bromide), tiotropium, atropine or oxitropium); hormones, (e.g., cortisone, hydrocortisone or prednisolone); xanthines, (e.g., aminophylline, choline theophyllinate, lysine theophyllinate or theophylline); therapeutic proteins and peptides, (e.g., insulin). In addition to those stated above, it will be clear to a person skilled in the art that, where appropriate, the medicaments may be used in the form of salts, (e.g., as alkali metal or amine salts or as acid addition salts) or as esters (e.g., lower alkyl esters) or as solvates (e.g., hydrates) to optimize the activity and/or stability of the medicament. It will be further clear to a person skilled in the art that where appropriate, the medicaments may be used in the form of a pure isomer, for example, R-salbutamol or RR-formoterol.

Particular medicaments for administration using pharmaceutical formulations in accordance with the invention include anti-allergics, bronchodilators, beta agonists (e.g., long-acting beta agonists), and anti-inflammatory steroids of use in the treatment of respiratory conditions, as defined herein, by inhalation therapy, for example, cromoglicate (e.g. as the sodium salt), salbutamol (e.g. as the free base or the sulphate salt), salmeterol (e.g. as the xinafoate salt), bitolterol, formoterol (e.g. as the fumarate salt), terbutaline (e.g. as the sulphate salt), 3-(4-{[6-({(2R)-2-hydroxy-2-[4-hydroxy-3-(hydroxymethyl)phenyl]ethyl}amino)hexyl]oxy}butyl)benzenesulfonamide, 3-(3-{[7-({(2R)-2-hydroxy-2-[4-hydroxy-3-(hydroxymethyl)phenyl]ethyl}amino)heptyl]oxy}propyl)benzenesulfonamide, 4-{(1R)-2-[(6-{2-[(2,6-dichlorobenzyl)oxy]ethoxy}hexyl)amino]-1-hydroxyethyl}-2-(hydroxymethyl)phenol, 2-hydroxy-5-((1R)-1-hydroxy-2-{[2-(4-{[(2R)-2-hydroxy-2-phenylethyl]amino}phenyl)ethyl]amino}ethyl)phenylformamide, 8-hydroxy-5-{(1R)-1-hydroxy-2-[(2-{4-[(6-methoxy-1,1′-biphenyl-3-yl)amino]phenyl}ethyl)amino]ethyl}quinolin-2(1H)-one, reproterol (e.g. as the hydrochloride salt), a beclomethasone ester (e.g. the dipropionate), a fluticasone ester (e.g. the propionate), a mometasone ester (e.g., the furoate), budesonide, dexamethasone, flunisolide, triamcinolone, tripredane, (22R)-6α,9α-difluoro-11β,21-dihydroxy-16α,17α-propylmethylenedioxy-4-pregnen-3,20-dione. Medicaments useful in erectile dysfunction treatment (e.g., PDE-V inhibitors such as vardenafil hydrochloride, along with alprostadil and sildenafil citrate) may also be employed. It should be understood that the medicaments that may be used in conjunction with the inhaler are not limited to those described herein.

Salmeterol, especially salmeterol xinafoate, salbutamol, fluticasone propionate, beclomethasone dipropionate and physiologically acceptable salts and solvates thereof are especially preferred.

It will be appreciated by those skilled in the art that the formulations according to the invention may, if desired, contain a combination of two or more medicaments. Formulations containing two active ingredients are known for the treatment and/or prophylaxis of respiratory disorders such as those described herein, for example, formoterol (e.g. as the fumarate) and budesonide, salmeterol (e.g. as the xinafoate salt) and fluticasone (e.g. as the propionate ester), salbutamol (e.g. as free base or sulphate salt) and beclomethasone (as the dipropionate ester) are preferred.

In one embodiment, a particular combination that may be employed is a combination of a beta agonist (e.g., a long-acting beta agonist) and an anti-inflammatory steroid. One embodiment encompasses a combination of salmeterol, or a salt thereof (particularly the xinafoate salt) and fluticasone propionate. The ratio of salmeterol to fluticasone propionate in the formulations according to the present invention is preferably within the range 4:1 to 1:20. The two drugs may be administered in various manners, simultaneously, sequentially, or separately, in the same or different ratios. In various embodiments, each metered dose or actuation of the inhaler will typically contain from 25 μg to 100 μg of salmeterol and from 25 μg to 500 μg of fluticasone propionate. The pharmaceutical formulation may be administered as a formulation according to various occurrences per day. In one embodiment, the pharmaceutical formulation is administered twice daily.

Embodiments of specific medicament combinations that may be used in various pharmaceutical formulations are as follows:

1) fluticasone propionate 100 μg/salmeterol 50 μg

2) fluticasone propionate 250 μg/salmeterol 50 μg

3) fluticasone propionate 500 μg/salmeterol 50 μg

In various embodiments, the pharmaceutical formulations may be present in the form of various inhalable formulations. In one embodiment, the pharmaceutical formulation is present in the form of a dry powder formulation, the formulation of such may be carried out according to known techniques. Dry powder formulations for topical delivery to the lung by inhalation may, for example, be presented in capsules and cartridges of, for example, gelatine, or blisters of, for example, laminated aluminum foil, for use in an inhaler or insufflator. Powder blend formulations generally contain a powder mix for inhalation of the compound of the invention and a suitable powder base which includes lactose and, optionally, at least one additional excipient (e.g., carrier, diluent, etc.). In various embodiments, each capsule or cartridge may generally contain between 20 μg and 10 mg of the at least one medicament. In one embodiment, the formulation may be formed into particles comprising at least one medicament, and excipient material(s), such as by co-precipitation or coating. When employed as a dry powder, packaging of the formulation may be suitable for unit dose or multi-dose delivery. In the case of multi-dose delivery, the formulation can be pre-metered (e.g., as in Diskus®, See GB 2242134/U.S. Pat. Nos. 6,032,666, 5,860,419, 5,873,360, 5,590,645, 6,378,519, 6,536,427, and 6,792,645 or Diskhaler, See GB 2178965, 2129691 and 2169265, U.S. Pat. Nos. 4,778,054, 4,811,731, 5,035,237) or metered in use (e.g. as in Turbuhaler, See EP 69715, or in the devices described in U.S. Pat. No. 6,321,747). An example of a unit-dose device is Rotahaler® (See GB 2064336). In one embodiment, the Diskus® inhalation device comprises an elongate strip formed from a base sheet having a plurality of recesses spaced along its length and a lid sheet hermetically but peelably sealed thereto to define a plurality of containers, each container having therein an inhalable formulation containing the at least one medicament, the lactose, optionally with other excipients. Preferably, the strip is sufficiently flexible to be wound into a roll. The lid sheet and base sheet will preferably have leading end portions which are not sealed to one another and at least one of the leading end portions is constructed to be attached to a winding means. Also, preferably the hermetic seal between the base and lid sheets extends over their whole width. The lid sheet may preferably be peeled from the base sheet in a longitudinal direction from a first end of the base sheet.

The pharmaceutical formulation formed by the processes of the invention may be used in the treatment of a number of respiratory disorders. Such respiratory conditions include, without limitation, diseases and conditions associated with reversible airways obstruction such as asthma, chronic obstructive pulmonary disease (e.g. chronic and wheezy bronchitis, emphysema), respiratory tract infection and upper respiratory tract disease (e.g. rhinitis, such as allergic and seasonal rhinitis). Such treatment is carried out by delivering medicament to a mammal. In will be appreciated by those skilled in the art that reference herein to “treatment” extends to prophylaxis as well as addressing established conditions. Accordingly, and in view of the above, in another aspect, the invention provides a method for the treatment of a respiratory disorder comprising the step of administering a pharmaceutically effective amount of a pharmaceutical formulation to a mammal such as, for example, a human. For the purposes of the invention, the term “pharmaceutically effective amount” is to be broadly interpreted and encompass the treatment of the disorder. In one embodiment, the administration is carried out via an inhalation device described herein. In one embodiment, the administration is carried out by nasal or oral inhalation.

The following examples are intended to illustrate the invention, and do not limit the scope of the invention as defined by the claims. In the examples X50 values are in microns (μm).

### APPENDIX 1

### Sympatec Methodology

Instrumental Parameters

TABLE 1

| Parameter | Typical |
|---|---|
| Measuring Range | R5: 0.5/4.5→875 μm |
| Trigger Conditions | 10 s, 100 ms, 0.2% ch12 |
| Time Base | 100 ms |
| Reference measurement | 10 s, Single |
| Focus Prior to first measurement | No |
| Start | 0 s after channel 12 ≧ 0.2% |
| Valid | Always |
| Stop | 5 s after channel 12 ≦ 0.2% |
| Or After | 30 s real time |
| Dispersing Device | RODOS |
| VIBRI Chute | Standard chute |
| Gap Width | 2 mm |
| Pressure | Nominal 1.5 Bar (1.3-1.7 Bar |
| | acceptable) |
| Depression | Maximise |
| Feed Rate | 85% |
| Sample Weight | 2 g ± 1 g |

The Sympatec HELOS is provided with the RODOS dry powder dispersion unit and the VIBRI vibratory feeder.

In these measurements, software is used in conjunction with the measurement equipment.

For the RODOS dispersing system, the primary pressure of the injector should be adjusted using the pressure control dial. The primary pressure should be within the range 1.3-1.7 bar although a pressure of 1.5 bar should be aimed for at each run. The injector depression should be optimised using the adjustment ring. The direction in which the adjuster ring is turned (clockwise or anti clockwise), has no adverse effect on the depression obtained. On instruments with a RODOS/M dispersing system, the primary pressure may be adjusted using a software algorithm. The injector depression should be maximised by clicking the “Auto-adjust depr” button. The instrument should not be used if the injector depression is less than 55 mbar at 1.3-1.7 bar.

### APPENDIX 2

### Malvern Operating Conditions and Method

TABLE 2
Equipment and Operating Conditions

| | |
|---|---|
| Instrument | Malvern Mastersizer 2000 |
| Software | Version 5.22 (not critical) |
| version | |
| Dispersant | 0.05% lecithin in iso-Octane |
| Background | Background and measurement sequence both 10 s |
| | (10,000 sweeps). All detectors less than 200 units, |
| | preferably less than 100 units |
| Pre- | For DCL Fine lactose |
| dispersion | Weigh 20 ± 5 mg into a 20 ml scintillation vial and add |
| | 15 ± 1 ml dispersant. Hand shake to prevent the |
| | sample forming a plug at the bottom of the vial. |
| | Sonicate for 30 ± 1 s in an ultrasonic bath. Add drop- |
| | wise to measuring cell to obtain required obscuration |
| | For DCL Coarse Lactose |
| | Weigh 150 ± 5 mg into a 20 ml scintillation vial and add |
| | 15 ± 1 ml dispersant. Hand shake to prevent the |
| | sample forming a plug at the bottom of the vial. Place |
| | the vial into a Whirlimixer (vortex mixer) and shake |
| | for 30 ± 1 s in. Add drop-wise to measuring cell to |
| | obtain required obscuration |
| Obscuration | 5-12% |
| Pump speed | 2500 ± 10 rpm |
| Optical model | Fraunhofer Theory |

### APPENDIX 3

Manufacture of engineered seed material using nanomilled seed (See e.g., U.S. co-pending patent application Ser. No. 60/821,871 entitled “Process for Manufacturing Lactose” filed concurrently herewith)

The procedure was performed in a HEL AutoMate reactor system (Hazard Automation Laboratories Barnet, London). 19.2 g of lactose (from Friesland Foods Domo, Netherlands) was dissolved in 18 ml of water at 85-90° C. The mixture was cooled to 50° C. and 12 ml of acetone added to the solution. The solution was seeded with an acetone suspension of nanomilled lactose having a median diameter of approximately 0.2 microns. (The dry weight of nanomilled lactose particles was 300 mg). The seeded mixture was then cooled to 20° C. at a rate of −1° C./min. The material was then filtered (how) and the solid washed with 50 ml of 40% acetone/water and the cake slurried in 2×50 ml of acetone. The solid was pulled dry for 30 min. The solid unloaded and dried in vacuo at 40° C. overnight. The particle size distribution was measured using Sympatec HELOS particle size analysis (See Appendix 1). X50 was 5.69.

### Example 1

### Crystallization Procedure

75 g of α-lactose monohydrate was dissolved in 225 ml of water and heated to 65° C. to form a solution. The solution was base treated with Amberlite IRA410 and stirred at room temperature for 45 min. pH increased from 3.35 to 8.95 over this 45 min period. Water was then distilled off in vacuo (161 ml) to leave a solution of lactose in 64 ml water. The solution was then heated to 95 C in an oil bath and 8 ml of water added. After 15 min at 95° C. the oil bath was set to 20° C. and the solution cooled. At 56° C. the mixtures was seeded with lactose (Friesland Foods Domo, Netherlands Lactohale Grade 3) and crystallization ensured. The material was allowed to cool in the oil bath overnight. The material was filtered off. The vessel was rinsed with 4 ml water and the rinse was filtered. The lactose was dried in vacuo to constant weight to give 47.2 of solid. The pH of the lactose in 25% solution was 7.09. The median particle size distribution of the crystallized lactose was 84 μm.

### Example 2

### Crystallization Procedure

32 g of lactose (from Friesland Foods Domo, Netherlands) was dissolved in 30 ml of water at 85-90° C. The pH of the solution was measured (pH 3.84). The mixture was cooled to 50° C. and the solution was seeded with 3.125 mg/g [mg of Seed/gram of input] microfine lactose (Batch “A”, Friesland Foods Domo, Netherlands, PSD; D-10, 0.85 μm; D-50, 3.21 μm; D-90, 7.74 μm). The solution was then cooled to 20° C. over 10 hours following an inverse cooling cooling profile defined by the equation T(t)=Tᵢ−(Tᵢ−T_{f})(t/t_{f})³, where T(t)=temperature at time t, Tᵢ=initial temperature, T_{f}=final temperature and t_{f}=batch time. The material was filtered and the solid washed with 50 ml of 20% ethanol/water, 50 ml of 40% ethanol/water and the cake slurried in ethanol. The solid was pulled dry for 1 hour and the solid unloaded and dried in vacuo (Gallenkamp vacuum oven) at 40° C. overnight. The particle size distribution was measured using Sympatec HELOS particle size analysis (See Appendix 1). X50 was 49.01.

### Example 3

### Crystallization Procedure

32 g of lactose (from Friesland Foods Domo, Netherlands) was dissolved in 30 ml of water at 85-90° C. The pH of the solution was measured (pH 6.9). The mixture was cooled to 50° C. and the solution was seeded with 3.125 mg/g [per g of input material] microfine lactose (Batch “A”, Friesland Foods Domo, Netherlands, PSD; D-10, 0.85 μm; D-50, 3.21 μm; D-90, 7.74 μm). The solution was then cooled to 20° C. over 5 hours at a rate of −0.133 C/min. The material was filtered and the solid washed with 50 ml of 20% ethanol/water, 50 ml of 40% ethanol/water and the cake slurried in ethanol. The solid was pulled dry for 1 hour in the Buchner filter funnel and the solid unloaded and dried in vacuo (Gallenkamp vacuum oven) at 40° C. overnight. The particle size distribution was measured using Sympatec HELOS particle size analysis (See Appendix 1). X50 was 28.

### Example 4

### Crystallization Procedure

32 g of lactose (Friesland Foods Domo, Netherlands) was dissolved in 30 ml of water at 85-90° C. The pH of the solution was measured (pH 4.1). The mixture was cooled to 50° C. and the solution was seeded with 3.437 mg/g [per g of input material] microfine lactose (Batch “A”, Friesland Foods Domo, Netherlands, PSD; D-10, 0.85 μm; D-50, 3.21 μm; D-90, 7.74 μm). The solution was then cooled to 20° C. over 10 hours using an inverse cooling method. The material was filtered and the solid washed with 50 ml of 20% ethanol/water, 50 ml of 40% ethanol/water and the cake slurried in ethanol. The solid was pulled dry for 1 hour (using Buchner filter funnel) and the solid unloaded and dried in vacuo (Gallenkamp vacuum oven) at 40° C. overnight. The particle size distribution was measured using Sympatec HELOS particle size analysis (See Appendix 1). X50 was 37.29.

### Example 5

### Crystallization Procedure

32 g of lactose (Friesland Foods Domo, Netherlands) was dissolved in 30 ml of water at 85-90° C. The pH of the solution was measured (pH 8.1). The mixture was cooled to 50° C. and the solution was seeded with 3.125 mg/g [per g of input material] microfine lactose (Batch “A”, Friesland Foods Domo, Netherlands, PSD; D-10, 0.85 μm; D-50, 3.21 μm; D-90, 7.74 μm). The solution was then cooled to 20° C. over 10 hours using an inverse cooling profile. The material was filtered and the solid washed with 50 ml of 20% ethanol/water, 50 ml of 40% ethanol/water and the cake slurried in ethanol. The solid was pulled dry for 1 hour (using Buchner filter funnel) and the solid unloaded and dried in vacuo (Gallenkamp vacuum oven) at 40° C. overnight. The particle size distribution was measured using Sympatec HELOS particle size analysis (See Appendix 1). X50 was 24.16.

### Example 6

### Crystallization Procedure

32 g of lactose (Friesland Foods Domo, Netherlands) was dissolved in 30 ml of water at 85-90° C. The pH of the solution was measured (pH 6.0). The mixture was cooled to 50° C. and the solution was seeded with 3.437 mg/g [per g of input material] microfine lactose (Batch “A”, Friesland Foods Domo, Netherlands, PSD; D-10, 0.85 μm; D-50, 3.21 μm; D-90, 7.74 μm). The solution was then cooled to 20° C. over 10 hours using an inverse cooling profile. The material was filtered and the solid washed with 50 ml of 20% ethanol/water, 50 ml of 40% ethanol/water and the cake slurried in ethanol. The solid was pulled dry for 1 hour (using Buchner filter funnel) and the solid unloaded and dried in vacuo (Gallenkamp vacuum oven) at 40° C. overnight. The particle size distribution was measured using Sympatec HELOS particle size analysis (See Appendix 1). X50 was 29.52.

### Example 7

### Crystallization Procedure

32 g of lactose (Friesland Foods Domo, Netherlands) was dissolved in 30 ml of water at 85-90° C. The pH of the solution was measured. 0.1 ml of 0.5M sodium hydroxide solution was added to the hot solution and the pH of the solution was measured again (pH 4.88). The mixture was cooled to 50° C. and the solution was seeded with 3.125 mg/g [per g of input material] microfine lactose (Batch “A”, Friesland Foods Domo, Netherlands, PSD; D-10, 0.85 μm; D-50, 3.21 μm; D-90, 7.74 μm). The solution was then cooled to 20° C. over 5 hours at a rate of −0.133 C/min. The material was filtered and the solid washed with 50 ml of 20% ethanol/water, 50 ml of 40% ethanol/water and the cake slurried in ethanol. The solid was pulled dry for 1 hour (using a buchner filter funnel) and the solid unloaded and dried in vacuo (Gallenkamp vacuum oven) at 40° C. overnight. The particle size distribution was measured using Sympatec HELOS particle size analysis (See Appendix 1). X50 was 32.15.

### Example 8

### Crystallization Procedure

32 g of lactose (Friesland Foods Domo, Netherlands) was dissolved in 30 ml of water at 85-90° C. The pH of the solution was measured. 0.1 ml of 0.5M sodium hydroxide was added to the hot solution and the pH of the solution was measured again (pH 5.13). The mixture was cooled to 50° C. and the solution was seeded with 3.125 mg/g [per g of input material] microfine lactose (Batch “A”, Friesland Foods Domo, Netherlands, PSD; D-10, 0.85 μm; D-50, 3.21 μm; D-90, 7.74 μm). The solution was then cooled to 20° C. over 5 hours at a rate of −0.133 C/min. The material was filtered and the solid washed with 50 ml of 20% ethanol/water, 50 ml of 40% ethanol/water and the cake slurried in ethanol. The solid was pulled dry for 1 hour (using what a Buchner filter funnel) and the solid unloaded and dried in vacuo (Gallenkamp vacuum oven) at 40° C. overnight. The particle size distribution was measured using Sympatec HELOS particle size analysis (See Appendix 1). X50 was 29.79.

### Example 9

### Crystallization Procedure

32 g of lactose (Lactose New Zealand, Hawera, New Zealand) was dissolved in 30 ml of water at 85-90° C. The pH of the solution was measured. 0.1 ml of 0.5M sodium hydroxide solution was added to the hot solution and the pH of the solution was measured again (pH 5.89). The mixture was cooled to 50° C. and the solution was seeded with 3.125 mg/g microfine lactose (Batch “A”, Friesland Foods Domo, Netherlands, PSD; D-10, 0.85 μm; D-50, 3.21 μm; D-90, 7.74 μm). The solution was then cooled to 20° C. over 5 hours at a rate of −0.133 C/min. The material was filtered and the solid washed with 50 ml of 20% ethanol/water, 50 ml of 40% ethanol/water and the cake slurried in ethanol. The solid was pulled dry for 1 hour (using a Buchner funnel) and the solid unloaded and dried in vacuo (Gallenkamp vacuum oven) at 40° C. overnight. The particle size distribution was measured using Sympatec HELOS particle size analysis (See Appendix 1). X50 was 32.2.

### Example 10

### Crystallization Procedure

32 g of lactose (Lactose New Zealand, Hawera, New Zealand) was dissolved in 30 ml of water at 85-90° C. The pH of the solution was measured. 0.1 ml of 0.5M sodium hydroxide solution was added to the hot solution and the pH of the solution was measured again (pH 6.36). The mixture was cooled to 50° C. and the solution was seeded with 3.125 mg/g [per g of input material] microfine lactose (Batch “A”, Friesland Foods Domo, Netherlands, PSD; D-10, 0.85 μm; D-50, 3.21 μm; D-90, 7.74 μm). The solution was then cooled to 20° C. over 5 hours at a rate of −0.133 C/min. The material was filtered and the solid washed with 50 ml of 20% ethanol/water, 50 ml of 40% ethanol/water and the cake slurried in ethanol. The solid was pulled dry for 1 hour (using a Buchner filter funnel) and the solid unloaded and dried in vacuo (Gallenkamp vacuum oven) at 40° C. overnight. The particle size distribution was measured using Sympatec HELOS particle size analysis (See Appendix 1). X50 was 30.91.

### Example 11

### Crystallization Procedure

32 g of lactose (Friesland Foods Domo, Netherlands) was dissolved in 30 ml of water at 85-90° C. The pH of the solution was measured. 0.2 ml of 0.5M sodium hydroxide solution was added to the hot solution and the pH of the solution was measured again (pH 6.27). The mixture was cooled to 50° C. and the solution was seeded with 3.125 mg/g microfine lactose (Batch “A”, Friesland Foods Domo, Netherlands, PSD; D-10, 0.85 μm; D-50, 3.21 μm; D-90, 7.74 μm). The solution was then cooled to 20° C. over 5 hours at a rate of −0.133 C/min. The material was filtered and the solid washed with 50 ml of 20% ethanol/water, 50 ml of 40% ethanol/water and the cake slurried in ethanol. The solid was pulled dry for 1 hour (using what Buchner filter funnel) and the solid unloaded and dried in vacuo (Gallenkamp vacuum oven) at 40° C. overnight. The particle size distribution was measured using Sympatec HELOS particle size analysis (See Appendix 1). X50 was 27.12.

### Example 12

### Crystallization Procedure

32 g of lactose (Friesland Foods Domo, Netherlands) was dissolved in 30 ml of water at 85-90° C. The pH of the solution was measured. 0.2 ml of 0.5M sodium hydroxide was added to the hot solution and the pH of the solution was measured again (pH 6.25). The mixture was cooled to 50° C. and the solution was seeded with 3.125 mg/g [per g of input material] microfine lactose (Batch “A”, Friesland Foods Domo, Netherlands, PSD; D-10, 0.85 μm; D-50, 3.21 μm; D-90, 7.74 μm). The solution was then cooled to 20° C. over 5 hours at a rate of −0.133 C/min. The material was filtered and the solid washed with 50 ml of 20% ethanol/water, 50 ml of 40% ethanol/water and the cake slurried in ethanol. The solid was pulled dry for 1 hour (using a Buchner filter funnel) and the solid unloaded and dried in vacuo (Gallenkamp vacuum oven) at 40° C. overnight. The particle size distribution was measured using Sympatec HELOS particle size analysis (See Appendix 1). X50 was 30.6.

### Example 13

### Crystallization Procedure

32 g of lactose (Lactose New Zealand, Hawera, New Zealand) was dissolved in 30 ml of water at 85-90° C. The pH of the solution was measured. 0.2 ml of 0.5M sodium hydroxide was added to the hot solution and the pH of the solution was measured again (pH 6.3). The mixture was cooled to 50° C. and the solution was seeded with 3.125 mg/g [per g of input material] microfine lactose (Batch “A”, Friesland Foods Domo, Netherlands, PSD; D-10, 0.85 μm; D-50, 3.21 μm; D-90, 7.74 μm). The solution was then cooled to 20° C. over 5 hours at a rate of −0.133 C/min. The material was filtered and the solid washed with 50 ml of 20% ethanol/water, 50 ml of 40% ethanol/water and the cake slurried in ethanol. The solid was pulled dry for 1 hour (using a Buchner filter funnel) and the solid unloaded and dried in vacuo (Gallenkamp vacuum oven) at 40° C. overnight. The particle size distribution was measured using Sympatec HELOS particle size analysis (See Appendix 1). X50 was 28.42.

### Example 14

### Crystallization Procedure

32 g of lactose (Lactose New Zealand, Hawera, New Zealand) was dissolved in 30 ml of water at 85-90° C. The pH of the solution was measured. 0.2 ml of 0.5M sodium hydroxide solution was added to the hot solution and the pH of the solution was measured again (pH 6.2). The mixture was cooled to 50° C. and the solution was seeded with 3.125 mg/g [per g of input material] microfine lactose (Batch “A”, Friesland Foods Domo, Netherlands, PSD; D-10, 0.85 μm; D-50, 3.21 μm; D-90, 7.74 μm). The solution was then cooled to 20° C. over 5 hours at a rate of −0.133 C/min. The material was filtered and the solid washed with 50 ml of 20% ethanol/water, 50 ml of 40% ethanol/water and the cake slurried in ethanol. The solid was pulled dry for 1 hour (using a Buchner filter) and the solid unloaded and dried in vacuo (Gallenkamp vacuum oven) at 40° C. overnight. The particle size distribution was measured using Sympatec HELOS particle size analysis (See Appendix 1). X50 was 27.9.

### Example 15

### Crystallization Procedure

32 g of lactose (Lactose New Zealand, Hawera, New Zealand) was dissolved in 30 ml of water at 85-90° C. The pH of the solution was measured. 0.3 ml of 0.5M sodium hydroxide was added to the hot solution and the pH of the solution was measured again (pH 5.07). The mixture was cooled to 50° C. and the solution was seeded with 3.125 mg/g [per g of input material] microfine lactose (Batch “A”, Friesland Foods Domo, Netherlands, PSD; D-10, 0.85 μm; D-50, 3.21 μm; D-90, 7.74 μm). The solution was then cooled to 20° C. over 5 hours at a rate of −0.133 C/min. The material was filtered and the solid washed with 50 ml of 20% ethanol/water, 50 ml of 40% ethanol/water and the cake slurried in ethanol. The solid was pulled dry for 1 hour (using a Buchner filter) and the solid unloaded and dried in vacuo (Gallenkamp vacuum oven) at 40° C. overnight. The particle size distribution was measured using Sympatec HELOS particle size analysis (See Appendix 1). X50 was 28.69.

### Example 16

### Crystallization Procedure

32 g of lactose (Lactose New Zealand, Hawera, New Zealand) was dissolved in 30 ml of water at 85-90° C. The pH of the solution was measured. 0.3 ml of 0.5M sodium hydroxide solution was added to the hot solution and the pH of the solution was measured again (pH 5.24). The mixture was cooled to 50° C. and the solution was seeded with 3.125 mg/g [per g of input material] microfine lactose (Batch “A”, Friesland Foods Domo, Netherlands, PSD; D-10, 0.85 μm; D-50, 3.21 μm; D-90, 7.74 μm). The solution was then cooled to 20° C. over 5 hours at a rate of −0.133 C/min. The material was filtered and the solid washed with 50 ml of 20% ethanol/water, 50 ml of 40% ethanol/water and the cake slurried in ethanol. The solid was pulled dry for 1 hour (using a Buchner filter funnel) and the solid unloaded and dried in vacuo (Gallenkamp vacuum oven) at 40° C. overnight. The particle size distribution was measured using Sympatec HELOS particle size analysis (See Appendix 1). X50 was 29.00.

### Example 17

### Crystallization Procedure

32 g of lactose (Friesland Foods Domo, Netherlands) was dissolved in 30 ml of water at 85-90° C. The pH of the solution was measured. 0.3 ml of 0.5M sodium hydroxide solution was added to the hot solution and the pH of the solution was measured again (pH 5.69). The mixture was cooled to 50° C. and the solution was seeded with 3.125 mg/g [per g of input material] microfine lactose (Batch “A”, Friesland Foods Domo, Netherlands, PSD; D-10, 0.85 μm; D-50, 3.21 μm; D-90, 7.74 μm). The solution was then cooled to 20° C. over 5 hours at a rate of −0.133 C/min. The material was filtered and the solid washed with 50 ml of 20% ethanol/water, 50 ml of 40% ethanol/water and the cake slurried in ethanol. The solid was pulled dry for 1 hour (using a Buchner filter) and the solid unloaded and dried in vacuo (Gallenkamp vacuum oven) at 40° C. overnight. The particle size distribution was measured using Sympatec HELOS particle size analysis (See Appendix 1). X50 was 28.43.

### Example 18

### Crystallization Procedure

32 g of lactose (Friesland Foods Domo, Netherlands) was dissolved in 30 ml of water at 85-90° C. The pH of the solution was measured. 0.3 ml of 0.5M sodium hydroxide solution was added to the hot solution and the pH of the solution was measured again (pH 5.82). The mixture was cooled to 50° C. and the solution was seeded with 3.125 mg/g [per g of input material] microfine lactose (Batch “A”, Friesland Foods Domo, Netherlands, PSD; D-10, 0.85 μm; D-50, 3.21 μm; D-90, 7.74 μm). The solution was then cooled to 20° C. over 5 hours at a rate of −0.133 C/min. The material was filtered and the solid washed with 50 ml of 20% ethanol/water, 50 ml of 40% ethanol/water and the cake slurried in ethanol. The solid was pulled dry for 1 hour (using what a Buchner filter) and the solid unloaded and dried in vacuo (Gallenkamp vacuum oven) at 40° C. overnight. The particle size distribution was measured using Sympatec HELOS particle size analysis (See Appendix 1). X50 was 29.42.

### Example 19

### Crystallization Procedure

32 g of lactose (Lactose New Zealand, Hawera, New Zealand) was dissolved in 30 ml of water at 85-90° C. The pH of the solution was measured. 0.3 ml of 0.5M sodium hydroxide solution was added to the hot solution and the pH of the solution was measured again (pH 7.81). The mixture was cooled to 50° C. and the solution was seeded with 3.125 mg/g [per g of input material] microfine lactose (Batch “A”, Friesland Foods Domo, Netherlands, PSD; D-10, 0.85 μm; D-50, 3.21 μm; D-90, 7.74 μm). The solution was then cooled to 20° C. over 5 hours at a rate of −0.133 C/min. The material was filtered and the solid washed with 50 ml of 20% ethanol/water, 50 ml of 40% ethanol/water and the cake slurried in ethanol. The solid was pulled dry for 1 hour (using what a Buchner filter) and the solid unloaded and dried in vacuo (Gallenkamp vacuum oven) at 40° C. overnight. The particle size distribution was measured using Sympatec HELOS particle size analysis (See Appendix 1). X50 was 26.81.

### Example 20

### Crystallization Procedure

32 g of lactose (Lactose New Zealand, Hawera, New Zealand) was dissolved in 30 ml of water at 85-90° C. The pH of the solution was measured. 0.3 ml of 0.5M sodium hydroxide solution was added to the hot solution and the pH of the solution was measured again (pH 7.3). The mixture was cooled to 50° C. and the solution was seeded with 3.125 mg/g [per g of input material] microfine lactose (Batch “A”, Friesland Foods Domo, Netherlands, PSD; D-10, 0.85 μm; D-50, 3.21 μm; D-90, 7.74 μm). The solution was then cooled to 20° C. over 5 hours at a rate of −0.133 C/min. The material was filtered and the solid washed with 50 ml of 20% ethanol/water, 50 ml of 40% ethanol/water and the cake slurried in ethanol. The solid was pulled dry for 1 hour (using a Buchner filter) and the solid unloaded and dried in vacuo (Gallenkamp vacuum oven) at 40° C. overnight. The particle size distribution was measured using Sympatec HELOS particle size analysis (See Appendix 1). X50 was 27.29.

### Example 21

### Crystallization Procedure

32 g of lactose (Lactose New Zealand, Hawera, New Zealand) was dissolved in 30 ml of water at 85-90° C. The pH of the solution was measured. 0.3 ml of 0.5 M sodium hydroxide solution was added to the hot solution and the pH of the solution was measured again (pH 5.83). The mixture was cooled to 50° C. and the solution was seeded with 3.125 mg/g [per g of input material] microfine lactose (Batch “A”, Friesland Foods Domo, Netherlands, PSD; D-10, 0.85 μm; D-50, 3.21 μm; D-90, 7.74 μm). The solution was then cooled to 20° C. over 5 hours at a rate of −0.133 C/min. The material was filtered and the solid washed with 50 ml of 20% ethanol/water, 50 ml of 40% ethanol/water and the cake slurried in ethanol. The solid was pulled dry for 1 hour (using a Buchner filter) and the solid unloaded and dried in vacuo (Gallenkamp vacuum oven) at 40° C. overnight. The particle size distribution was measured using Sympatec HELOS particle size analysis (See Appendix 1). X50 was 28.66.

### Example 22

### Crystallization Procedure

32 g of lactose (Lactose New Zealand, Hawera, New Zealand) was dissolved in 30 ml of water at 85-90° C. The pH of the solution was measured. 0.3 ml of 0.5M sodium hydroxide solution was added to the hot solution and the pH of the solution was measured again (pH 5.07). The mixture was cooled to 50° C. and the solution was seeded with 3.125 mg/g [per g of input material] microfine lactose (Batch “A”, Friesland Foods Domo, Netherlands, PSD; D-10, 0.85 μm; D-50, 3.21 μm; D-90, 7.74 μm). The solution was then cooled to 20° C. over 5 hours at a rate of −0.133 C/min. The material was filtered and the solid washed with 50 ml of 20% ethanol/water, 50 ml of 40% ethanol/water and the cake slurried in ethanol. The solid was pulled dry for 1 hour (using a Buchner filter) and the solid unloaded and dried in vacuo (Gallenkamp vacuum oven) at 40° C. overnight. The particle size distribution was measured using Sympatec HELOS particle size analysis (See Appendix 1). X50 was 28.84.

### Example 23

### Crystallization Procedure

32 g of lactose (Friesland Foods Domo, Netherlands) was dissolved in 30 ml of water at 85-90° C. The pH of the solution was measured. 0.1 ml of 0.5 M sodium hydroxide solution was added to the hot solution and the pH of the solution was measured again (pH 5.26). The mixture was cooled to 50° C. and the solution was seeded with 3.125 mg/g [per g of input material] microfine lactose (Batch “A”, Friesland Foods Domo, Netherlands, PSD; D-10, 0.85 μm; D-50, 3.21 μm; D-90, 7.74 μm). The solution was then cooled to 20° C. over 10 hours using an inverse cooling profile. The material was filtered and the solid washed with 50 ml of 20% ethanol/water, 50 ml of 40% ethanol/water and the cake slurried in ethanol. The solid was pulled dry for 1 hour (using a Buchner filter) and the solid unloaded and dried in vacuo (Gallenkamp vacuum oven) at 40° C. overnight. The particle size distribution was measured using Sympatec HELOS particle size analysis (See Appendix 1). X50 was 29.25.

### Example 24

### Crystallization Procedure

32 g of lactose (Friesland Foods Domo, Netherlands) was dissolved in 30 ml of water at 85-90° C. The pH of the solution was measured. 0.2 ml of 0.5 M sodium hydroxide solution was added to the hot solution and the pH of the solution was measured again (pH 6.36). The mixture was cooled to 50° C. and the solution was seeded with 3.125 mg/g [per g of input material] microfine lactose (Batch “A”, Friesland Foods Domo, Netherlands, PSD; D-10, 0.85 μm; D-50, 3.21 μm; D-90, 7.74 μm). The solution was then cooled to 20° C. over 10 hours using an inverse cooling profile. The material was filtered and the solid washed with 50 ml of 20% ethanol/water, 50 ml of 40% ethanol/water and the cake slurried in ethanol. The solid was pulled dry for 1 hour (using a Buchner filter) and the solid unloaded and dried in vacuo (Gallenkamp vacuum oven) at 40° C. overnight. The particle size distribution was measured using Sympatec HELOS particle size analysis. X50 was 29.16.

### Example 25

### Crystallization Procedure

32 g of lactose (Friesland Foods Domo, Netherlands) was dissolved in 30 ml of water at 85-90° C. The pH of the solution was measured. 0.3 ml of 0.5 M sodium hydroxide solution was added to the hot solution and the pH of the solution was measured again (pH 7.15). The mixture was cooled to 50° C. and the solution was seeded with 3.125 mg/g [per g of input material] microfine lactose (Batch “A”, Friesland Foods Domo, Netherlands, PSD; D-10, 0.85 μm; D-50, 3.21 μm; D-90, 7.74 μm). The solution was then cooled to 20° C. over 10 hours using an inverse cooling profile. The material was filtered and the solid washed with 50 ml of 20% ethanol/water, 50 ml of 40% ethanol/water and the cake slurried in ethanol. The solid was pulled dry for 1 hour (using a Buchner filter) and the solid unloaded and dried in vacuo (Gallenkamp vacuum oven) at 40° C. overnight. The particle size distribution was measured using Sympatec HELOS particle size analysis (See Appendix 1). X50 was 28.52.

### Example 26

### Crystallization Procedure

32 g of lactose (Friesland Foods Domo, Netherlands) was dissolved in 30 ml of water at 85-90° C. The pH of the solution was measured. 0.4 ml of 0.5 M sodium hydroxide solution was added to the hot solution and the pH of the solution was measured again (pH 7.48). The mixture was cooled to 50° C. and the solution was seeded with 3.125 mg/g [per g of input material] microfine lactose (Batch “A”, Friesland Foods Domo, Netherlands, PSD; D-10, 0.85 μm; D-50, 3.21 μm; D-90, 7.74 μm). The solution was then cooled to 20° C. over 10 hours using an inverse cooling profile. The material was filtered and the solid washed with 50 ml of 20% ethanol/water, 50 ml of 40% ethanol/water and the cake slurried in ethanol. The solid was pulled dry for 1 hour (using a Buchner filter) and the solid unloaded and dried in vacuo (Gallenkamp vacuum oven) at 40° C. overnight. The particle size distribution was measured using Sympatec HELOS particle size analysis (See Appendix 1). X50 was 29.55.

### Example 27

### Crystallization Procedure

32 g of lactose (from Friesland Foods Domo, Netherlands) was dissolved in 30 ml of water at 85-90° C. The pH of the solution was measured (pH 6.9). The mixture was cooled to 50° C. and the solution was seeded with 1.25 mg/g [per g of input material] microfine lactose (Batch “A”, Friesland Foods Domo, Netherlands, PSD; D-10, 0.85 μm; D-50, 3.21 μm; D-90, 7.74 μm). The solution was then cooled to 20° C. over 10 hours using an inverse cooling profile. The material was filtered and the solid washed with 50 ml of 20% ethanol/water, 50 ml of 40% ethanol/water and the cake slurried in ethanol. The solid was pulled dry for 1 hour (using a Buchner filter funnel) and the solid unloaded and dried in vacuo (Gallenkamp vacuum oven) at 40° C. overnight. The particle size distribution was measured using Sympatec HELOS particle size analysis (See Appendix 1). X50 was 38.53.

### Example 28

### Crystallization Procedure

32 g of lactose (Friesland Foods Domo, Netherlands) was dissolved in 30 ml of water at 85-90° C. The pH of the solution was measured (pH 4.2). The mixture was cooled to 50° C. and the solution was seeded with 1.25 mg/g [per g of input material] microfine lactose (Batch “A”, Friesland Foods Domo, Netherlands, PSD; D-10, 0.85 μm; D-50, 3.21 μm; D-90, 7.74 μm). The solution was then cooled to 20° C. over 10 hours using an inverse cooling profile. The material was filtered and the solid washed with 50 ml of 20% ethanol/water, 50 ml of 40% ethanol/water and the cake slurried in ethanol. The solid was pulled dry for 1 hour (using a Buchner filter funnel) and the solid unloaded and dried in vacuo (Gallenkamp vacuum oven) at 40° C. overnight. The particle size distribution was measured using Sympatec HELOS particle size analysis (See Appendix 1). X50 was 45.61.

### Example 29

### Crystallization Procedure

32 g of lactose (Friesland Foods Domo, Netherlands) was dissolved in 30 ml of water at 85-90° C. The pH of the solution was measured (pH 8.0). The mixture was cooled to 50° C. and the solution was seeded with 1.25 mg/g [per g of input material] microfine lactose (Batch “A”, Friesland Foods Domo, Netherlands, PSD; D-10, 0.85 μm; D-50, 3.21 μm; D-90, 7.74 μm). The solution was then cooled to 20° C. over 10 hours using an inverse cooling profile. The material was filtered and the solid washed with 50 ml of 20% ethanol/water, 50 ml of 40% ethanol/water and the cake slurried in ethanol. The solid was pulled dry for 1 hour (using a Buchner filter funnel) and the solid unloaded and dried in vacuo (Gallenkamp vacuum oven) at 40° C. overnight. The particle size distribution was measured using Sympatec HELOS particle size analysis (See Appendix 1). X50 was 35.05.

### Example 30

### Crystallization Procedure

32 g of lactose (Friesland Foods Domo, Netherlands) was dissolved in 30 ml of water at 85-90° C. The pH of the solution was measured (pH 6.0). The mixture was cooled to 50° C. and the solution was seeded with 1.25 mg/g [per g of input material] microfine lactose (Batch “A”, Friesland Foods Domo, Netherlands, PSD; D-10, 0.85 μm; D-50, 3.21 μm; D-90, 7.74 μm). The solution was then cooled to 20° C. over 10 hours using an inverse cooling profile. The material was filtered and the solid washed with 50 ml of 20% ethanol/water, 50 ml of 40% ethanol/water and the cake slurried in ethanol. The solid was pulled dry for 1 hour (using a Buchner filter funnel) and the solid unloaded and dried in vacuo (Gallenkamp vacuum oven) at 40° C. overnight. The particle size distribution was measured using Sympatec HELOS particle size analysis (See Appendix 1). X50 was 38.54.

### Example 31

### Crystallization Procedure

32 g of lactose (Friesland Foods Domo, Netherlands) was dissolved in 30 ml of water at 85-90° C. The pH of the solution was measured. 0.1 ml of 0.5 M sodium hydroxide solution was added to the hot solution and the pH of the solution was measured again (pH 5.61). The mixture was cooled to 50° C. and the solution was seeded with 1.25 mg/g [per g of input material] microfine lactose (Batch “A”, Friesland Foods Domo, Netherlands, PSD; D-10, 0.85 μm; D-50, 3.21 μm; D-90, 7.74 μm). The solution was then cooled to 20° C. over 10 hours using an inverse cooling profile. The material was filtered and the solid washed with 50 ml of 20% ethanol/water, 50 ml of 40% ethanol/water and the cake slurried in ethanol. The solid was pulled dry for 1 hour (using a Buchner filter) and the solid unloaded and dried in vacuo (Gallenkamp vacuum oven) at 40° C. overnight. The particle size distribution was measured using Sympatec HELOS particle size analysis (See Appendix 1). X50 was 36.51.

### Example 32

### Crystallization Procedure

32 g of lactose (from Friesland Foods Domo, Netherlands) was dissolved in 30 ml of water at 85-90° C. The pH of the solution was measured. 0.2 ml of 0.5 M sodium hydroxide solution was added to the hot solution and the pH of the solution was measured again (pH 6.65). The mixture was cooled to 50° C. and the solution was seeded with 1.25 mg/g [per g of input material] microfine lactose (Batch “A”, Friesland Foods Domo, Netherlands, PSD; D-10, 0.85 μm; D-50, 3.21 μm; D-90, 7.74 μm). The solution was then cooled to 20° C. over 10 hours using an inverse cooling profile. The material was filtered and the solid washed with 50 ml of 20% ethanol/water, 50 ml of 40% ethanol/water and the cake slurried in ethanol. The solid was pulled dry for 1 hour (using a Buchner filter) and the solid unloaded and dried in vacuo (Gallenkamp vacuum oven) at 40° C. overnight. The particle size distribution was measured using Sympatec HELOS particle size analysis (See Appendix 1). X50 was 34.84.

### Example 33

### Crystallization Procedure

32 g of lactose (Friesland Foods Domo, Netherlands) was dissolved in 30 ml of water at 85-90° C. The pH of the solution was measured. 0.3 ml of 0.5 M sodium hydroxide solution was added to the hot solution and the pH of the solution was measured again (pH 7.25). The mixture was cooled to 50° C. and the solution was seeded with 1.25 mg/g [per g of input material] microfine lactose (Batch “A”, Friesland Foods Domo, Netherlands, PSD; D-10, 0.85 μm; D-50, 3.21 μm; D-90, 7.74 μm). The solution was then cooled to 20° C. over 10 hours using an inverse cooling profile. The material was filtered and the solid washed with 50 ml of 20% ethanol/water, 50 ml of 40% ethanol/water and the cake slurried in ethanol. The solid was pulled dry for 1 hour (using a Buchner filter) and the solid unloaded and dried in vacuo (Gallenkamp vacuum oven) at 40° C. overnight. The particle size distribution was measured using Sympatec HELOS particle size analysis (See Appendix 1). X50 was 34.95.

### Example 34

### Crystallization Procedure

32 g of lactose (Friesland Foods Domo, Netherlands) was dissolved in 30 ml of water at 85-90° C. The pH of the solution was measured. 0.4 ml of 0.5 M sodium hydroxide solution was added to the hot solution and the pH of the solution was measured again (pH 7.54). The mixture was cooled to 50° C. and the solution was seeded with 1.25 mg/g [per g of input material] microfine lactose (Batch “A”, Friesland Foods Domo, Netherlands, PSD; D-10, 0.85 μm; D-50, 3.21 μm; D-90, 7.74 μm). The solution was then cooled to 20° C. over 10 hours using an inverse cooling profile. The material was filtered and the solid washed with 50 ml of 20% ethanol/water, 50 ml of 40% ethanol/water and the cake slurried in ethanol. The solid was pulled dry for 1 hour (using a Buchner filter) and the solid unloaded and dried in vacuo (Gallenkamp vacuum oven) at 40° C. overnight. The particle size distribution was measured using Sympatec HELOS particle size analysis (See Appendix 1). X50 was 34.58.

### Example 35

### Crystallization Procedure

32 g of lactose (Friesland Foods Domo, Netherlands) was dissolved in 30 ml of water at 85-90° C. The pH of the solution was measured (pH 3.7). The mixture was cooled to 50° C. and the solution was seeded with 0.625 mg/g [per g of input material] microfine lactose (Batch “A”, Friesland Foods Domo, Netherlands, PSD; D-10, 0.85 μm; D-50, 3.21 μm; D-90, 7.74 μm). The solution was then cooled to 20° C. over 10 hours using an inverse cooling profile. The material was filtered and the solid washed with 50 ml of 20% ethanol/water, 50 ml of 40% ethanol/water and the cake slurried in ethanol. The solid was pulled dry for 1 hour (using a Buchner filter) and the solid unloaded and dried in vacuo (Gallenkamp vacuum oven) at 40° C. overnight. The particle size distribution was measured using Sympatec HELOS particle size analysis (See Appendix 1). X50 was 56.75.

### Example 36

### Crystallization Procedure

32 g of lactose (Friesland Foods Domo, Netherlands) was dissolved in 30 ml of water at 85-90° C. The pH of the solution was measured (pH 7.1). The mixture was cooled to 50° C. and the solution was seeded with 0.625 mg/g [per g of input material] microfine lactose (Batch “A”, Friesland Foods Domo, Netherlands, PSD; D-10, 0.85 μm; D-50, 3.21 μm; D-90, 7.74 μm). The solution was then cooled to 50° C. over 10 hours using an inverse cooling profile. The material was filtered and the solid washed with 50 ml of 20% ethanol/water, 50 ml of 40% ethanol/water and the cake slurried in ethanol. The solid was pulled dry for 1 hour (using a Buchner filter funnel) and the solid unloaded and dried in vacuo (Gallenkamp vacuum oven) at 40° C. overnight. The particle size distribution was measured using Sympatec HELOS particle size analysis (See Appendix 1). X50 was 41.29.

### Example 37

### Crystallization Procedure

32 g of lactose (Friesland Foods Domo, Netherlands) was dissolved in 30 ml of water at 85-90° C. The pH of the solution was measured (pH 8.0). The mixture was cooled to 50° C. and the solution was seeded with 0.625 mg/g [per g of input material] microfine lactose (Batch “A”, Friesland Foods Domo, Netherlands, PSD; D-10, 0.85 μm; D-50, 3.21 μm; D-90, 7.74 μm). The solution was then cooled to 20° C. over 10 hours using an inverse cooling profile. The material was filtered and the solid washed with 50 ml of 20% ethanol/water, 50 ml of 40% ethanol/water and the cake slurried in ethanol. The solid was pulled dry for 1 hour (using a Buchner filter funnel) and the solid unloaded and dried in vacuo (Gallenkamp vacuum oven) at 40° C. overnight. The particle size distribution was measured using Sympatec HELOS particle size analysis (See Appendix 1). X50 was 40.72.

### Example 38

### Crystallization Procedure

32 g of lactose (Friesland Foods Domo, Netherlands) was dissolved in 30 ml of water at 85-90° C. The pH of the solution was measured. 0.1 ml of 0.5 M sodium hydroxide solution was added to the hot solution and the pH of the solution was measured again (pH 4.61). The mixture was cooled to 50° C. and the solution was seeded with 0.625 mg/g [per g of input material] microfine lactose (Batch “A”, Friesland Foods Domo, Netherlands, PSD; D-10, 0.85 μm; D-50, 3.21 μm; D-90, 7.74 μm). The solution was then cooled to 20° C. over 10 hours using an inverse cooling profile. The material was filtered and the solid washed with 50 ml of 20% ethanol/water, 50 ml of 40% ethanol/water and the cake slurried in ethanol. The solid was pulled dry for 1 hour (using a Buchner filter) and the solid unloaded and dried in vacuo (Gallenkamp vacuum oven) at 40° C. overnight. The particle size distribution was measured using Sympatec HELOS particle size analysis (See Appendix 1). X50 was 44.73.

### Example 39

### Crystallization Procedure

32 g of lactose (Friesland Foods Domo, Netherlands) was dissolved in 30 ml of water at 85-90° C. The pH of the solution was measured. 0.2 ml of 0.5 M sodium hydroxide solution was added to the hot solution and the pH of the solution was measured again (pH 7.00). The mixture was cooled to 50° C. and the solution was seeded with 0.625 mg/g [per g of input material] microfine lactose (Batch “A”, Friesland Foods Domo, Netherlands, PSD; D-10, 0.85 μm; D-50, 3.21 μm; D-90, 7.74 μm). The solution was then cooled to 20° C. over 10 hours using an inverse cooling profile. The material was filtered and the solid washed with 50 ml of 20% ethanol/water, 50 ml of 40% ethanol/water and the cake slurried in ethanol. The solid was pulled dry for 1 hour (using a Buchner filter) and the solid unloaded and dried in vacuo (Gallenkamp vacuum oven) at 40° C. overnight. The particle size distribution was measured using Sympatec HELOS particle size analysis (See Appendix 1). X50 was 43.61.

### Example 40

### Crystallization Procedure

32 g of lactose (Friesland Foods Domo, Netherlands) was dissolved in 30 ml of water at 85-90° C. The pH of the solution was measured. 0.3 ml of 0.5 M sodium hydroxide solution was added to the hot solution and the pH of the solution was measured again (pH 7.32). The mixture was cooled to 50° C. and the solution was seeded with 0.625 mg/g [per g of input material] microfine lactose (Batch “A”, Friesland Foods Domo, Netherlands, PSD; D-10, 0.85 μm; D-50, 3.21 μm; D-90, 7.74 μm). The solution was then cooled to 20° C. over 10 hours using an inverse cooling profile. The material was filtered and the solid washed with 50 ml of 20% ethanol/water, 50 ml of 40% ethanol/water and the cake slurried in ethanol. The solid was pulled dry for 1 hour (using a Buchner filter) and the solid unloaded and dried in vacuo (Gallenkamp vacuum oven) at 40° C. overnight. The particle size distribution was measured using Sympatec HELOS particle size analysis (See Appendix 1). X50 was 43.07.

### Example 41

### Crystallization Procedure

32 g of lactose (Friesland Foods Domo, Netherlands) was dissolved in 30 ml of water at 85-90° C. The pH of the solution was measured. 0.4 ml of 0.5 M sodium hydroxide solution was added to the hot solution and the pH of the solution was measured again (pH 7.5). The mixture was cooled to 50° C. and the solution was seeded with 0.625 mg/g [per g of input material] microfine lactose (Batch “A”, Friesland Foods Domo, Netherlands, PSD; D-10, 0.85 μm; D-50, 3.21 μm; D-90, 7.74 μm). The solution was then cooled to 20° C. over 10 hours using an inverse cooling profile. The material was filtered and the solid washed with 50 ml of 20% ethanol/water, 50 ml of 40% ethanol/water and the cake slurried in ethanol. The solid was pulled dry for 1 hour (using a Buchner filter) and the solid unloaded and dried in vacuo (Gallenkamp vacuum oven) at 40° C. overnight. The particle size distribution was measured using Sympatec HELOS particle size analysis (See Appendix 1). X50 was 43.21.

### Example 42

### Crystallization Procedure

32 g of lactose (Friesland Foods Domo, Netherlands) was dissolved in 30 ml of water at 85-90° C. The pH of the solution was measured (pH 3.93). The mixture was cooled to 50° C. and the solution was seeded with 0.313 mg/g [per g of input material] microfine lactose (Batch “A”, Friesland Foods Domo, Netherlands, PSD; D-10, 0.85 μm; D-50, 3.21 μm; D-90, 7.74 μm). The solution was then cooled to 20° C. over 10 hours using an inverse cooling profile. The material was filtered and the solid washed with 50 ml of 20% ethanol/water, 50 ml of 40% ethanol/water and the cake slurried in ethanol. The solid was pulled dry for 1 hour (using a Buchner filter funnel) and the solid unloaded and dried in vacuo (Gallenkamp vacuum oven) at 40° C. overnight. The particle size distribution was measured using Sympatec HELOS particle size analysis (See Appendix 1). X50 was 56.78.

### Example 43

### Crystallization Procedure

32 g of lactose (Friesland Foods Domo, Netherlands) was dissolved in 30 ml of water at 85-90° C. The pH of the solution was measured (pH 7). The mixture was cooled to 50° C. and the solution was seeded with 0.313 mg/g [per g of input material] microfine lactose (Batch “A”, Friesland Foods Domo, Netherlands, PSD; D-10, 0.85 μm; D-50, 3.21 μm; D-90, 7.74 μm). The solution was then cooled to 20° C. over 10 hours using an inverse cooling profile. The material was filtered and the solid washed with 50 ml of 20% ethanol/water, 50 ml of 40% ethanol/water and the cake slurried in ethanol. The solid was pulled dry for 1 hour (using a Buchner funnel) and the solid unloaded and dried in vacuo (Gallenkamp vacuum oven) at 40° C. overnight. The particle size distribution was measured using Sympatec HELOS particle size analysis (See Appendix 1). X50 was 48.59.

### Example 44

### Crystallization Procedure

32 g of lactose (Friesland Foods Domo, Netherlands) was dissolved in 30 ml of water at 85-90° C. The pH of the solution was measured (pH 8.0). The mixture was cooled to 50° C. and the solution was seeded with 0.313 mg/g [per g of input material] microfine lactose (Batch “A”, Friesland Foods Domo, Netherlands, PSD; D-10, 0.85 μm; D-50, 3.21 μm; D-90, 7.74 μm). The solution was then cooled to 20° C. over 10 hours using an inverse cooling profile. The material was filtered and the solid washed with 50 ml of 20% ethanol/water, 50 ml of 40% ethanol/water and the cake slurried in ethanol. The solid was pulled dry for 1 hour (using a Buchner filter funnel) and the solid unloaded and dried in vacuo (Gallenkamp vacuum oven) at 40° C. overnight. The particle size distribution was measured using Sympatec HELOS particle size analysis (See Appendix 1). X50 was 47.11.

### Example 45

### Crystallization Procedure

32 g of lactose (Friesland Foods Domo, Netherlands) was dissolved in 30 ml of water at 85-90° C. The pH of the solution was measured. 0.3 ml of 0.5 M sodium hydroxide solution was added to the hot solution and the pH of the solution was measured again (pH 7.37). The mixture was cooled to 50° C. and the solution was seeded with 0.313 mg/g [per g of input material] microfine lactose (Batch “A”, Friesland Foods Domo, Netherlands, PSD; D-10, 0.85 μm; D-50, 3.21 μm; D-90, 7.74 μm). The solution was then cooled to 20° C. over 10 hours using an inverse cooling profile. The material was filtered and the solid washed with 50 ml of 20% ethanol/water, 50 ml of 40% ethanol/water and the cake slurried in ethanol. The solid was pulled dry for 1 hour (using a Buchner filter) and the solid unloaded and dried in vacuo (Gallenkamp vacuum oven) at 40° C. overnight. The particle size distribution was measured using Sympatec HELOS particle size analysis (See Appendix 1). X50 was 55.52.

### Example 46

### Crystallization Procedure

32 g of lactose (Friesland Foods Domo, Netherlands) was dissolved in 30 ml of water at 85-90° C. The pH of the solution was measured. 0.4 ml of 0.5 M sodium hydroxide was added to the hot solution and the pH of the solution was measured again (pH 7.73). The mixture was cooled to 50° C. and the solution was seeded with 0.313 mg/g [per g of input material] microfine lactose (Batch “A”, Friesland Foods Domo, Netherlands, PSD; D-10, 0.85 μm; D-50, 3.21 μm; D-90, 7.74 μm). The solution was then cooled to 20° C. over 10 hours using an inverse cooling profile. The material was filtered and the solid washed with 50 ml of 20% ethanol/water, 50 ml of 40% ethanol/water and the cake slurried in ethanol. The solid was pulled dry for 1 hour (using a Buchner filter) and the solid unloaded and dried in vacuo (Gallenkamp vacuum oven) at 40° C. overnight. The particle size distribution was measured using Sympatec HELOS particle size analysis (See Appendix 1). X50 was 55.45.

### Example 47

### Crystallization Procedure

The procedure was performed in a HEL AutoMate reactor system (Hazard Evaluation Laboratories Barnet, London) 32 g of lactose (Friesland Foods Domo, Netherlands) was dissolved in 30 ml of water at 85-90° C. Sodium hydroxide solution (0.5 N, 0.3 ml) was added to the hot solution. The mixture was cooled to 50° C. and the solution was seeded with 20 mg of seed (Batch “A”; Friesland Foods Domo, Netherlands) then cooled to 20° C. over 10 hours using an inverse cooling profile described by the equation T(t)=Tᵢ−(Tᵢ−T_{f})(t/t_{f})³, where T(t)=temperature at time t, tᵢ=initial temperature, T_{f}=final temperature and t_{f}=batch time. The material was then filtered using a Buchner filter and the solid washed with 30 ml of 40% acetone/water and the cake slurried in 2×50 ml of acetone. The solid was pulled dry for 30 min. The solid unloaded and dried in vacuo at 40° C. overnight. The particle size distribution was measured using Sympatec HELOS particle size analysis (See Appendix 1). X50 was 46.82.

### Example 48

### Crystallization Procedure

The procedure was performed in a HEL AutoMate reactor system (Hazard Evaluation Laboratories Barnet, London). 32 g of lactose (Friesland Foods Domo, Netherlands) was dissolved in 30 ml of water at 85-90° C. Sodium hydroxide solution (0.5 N, 0.3 ml) was added to the hot solution. The mixture was cooled to 50° C. and the solution was seeded with 20 mg of seed (Batch “B”; Friesland Foods Domo, Netherlands) then cooled to 20° C. over 10 hours using an inverse cooling profile described by the equation T(t)=Tᵢ−(Tᵢ−T_{f})(t/t_{f})³, where T(t)=temperature at time t, tᵢ=initial temperature, T_{f}=final temperature and t_{f}=batch time. The material was then filtered using a Buchner filter and the solid washed with 30 ml of 40% acetone/water and the cake slurried in 2×50 ml of acetone. The solid was pulled dry for 30 min. The solid unloaded and dried in vacuo at 40° C. overnight. The particle size distribution was measured using Sympatec HELOS particle size analysis (See Appendix 1). X50 was 25.94.

### Example 49

### Crystallization Procedure

The procedure was performed in a HEL AutoMate reactor system (Hazard Evaluation Laboratories Barnet, London). 32 g of lactose (Friesland Foods Domo, Netherlands) was dissolved in 30 ml of water at 85-90° C. Sodium hydroxide solution (0.5 N, 0.3 ml) was added to the hot solution. The mixture was cooled to 50° C. and the solution was seeded with 20 mg of seed (Batch “C”; Friesland Foods Domo, Netherlands) then cooled to 20° C. over 10 hours using an inverse cooling profile described by the equation T(t)=Tᵢ−(Tᵢ−T_{f})(t/t_{f})³, where T(t)=temperature at time t, tᵢ=initial temperature, T_{f}=final temperature and t_{f}=batch time. The material was then filtered (how) and the solid washed with 30 ml of 40% acetone/water and the cake slurried in 2×50 ml of acetone. The solid was pulled dry for 30 min. The solid unloaded and dried in vacuo at 40° C. overnight. The particle size distribution was measured using Sympatec HELOS particle size analysis (See Appendix 1). X50 was 39.32.

### Example 50

### Crystallization Procedure

The procedure was performed in a HEL AutoMate reactor system (Hazard Evaluation Laboratories Barnet, London). 32 g of lactose (Friesland Foods Domo, Netherlands) was dissolved in 30 ml of water at 85-90° C. Sodium hydroxide solution (0.5 N, 0.3 ml) was added to the hot solution. The mixture was cooled to 50° C. and the solution was seeded with 20 mg of engineered seed [See Appendix 3; X50—5.69] then cooled to 20° C. over 10 hours using an inverse cooling profile described by the equation T(t)=Tᵢ−(Tᵢ−T_{f})(t/t_{f})³, where T(t)=temperature at time t, tᵢ=initial temperature, T_{f}=final temperature and t_{f}=batch time. The material was then filtered (how) and the solid washed with 30 ml of 40% acetone/water and the cake slurried in 2×50 ml of acetone. The solid was pulled dry for 30 min. The solid unloaded and dried in vacuo at 40° C. overnight. The particle size distribution was measured using Sympatec HELOS particle size analysis (See Appendix 1). X50 was 45.54.

### Example 51

### Crystallization Procedure

The procedure was performed in a HEL AutoMate reactor system (Hazard Evaluation Laboratories Barnet, London). 32 g of lactose (Friesland Foods Domo, Netherlands) was dissolved in 30 ml of deionised water at 85-90° C. Sodium hydroxide solution (0.5 N, 0.3 ml) was added to the hot solution. The mixture was cooled to 50° C. and the solution was seeded with 1.25 mg/g of micronised seed (Batch “B”, Friesland Foods Domo, Netherlands). The mixture was cooled to 20° C. over 10 hours using an inverse cooling profile described by the equation T(t)=Tᵢ−(Tᵢ−T_{f})(t/t_{f})³, where T(t)=temperature at time t, tᵢ=initial temperature, T_{f}=final temperature and t_{f}=batch time. The crystallized material was then filtered using a Buchner filter and the solid washed with 30 ml of 40% acetone/water and then with 30 ml of acetone. The wet solid was slurred in 2×50 ml of acetone, filtered and air dried. The dried solid was further dried in vacuo at 40° C. overnight. The particle size distribution was measured using Sympatec HELOS particle size analysis (See Appendix 1). X50 was 33.53.

### Example 52

### Crystallization Procedure

The procedure was performed in a HEL AutoMate reactor system (Hazard Evaluation Laboratories Barnet, London). 32 g of lactose (Friesland Foods Domo, Netherlands) was dissolved in 30 ml of deionised water at 85-90° C. Sodium hydroxide solution (0.5 N, 0.3 ml) was added to the hot solution. The mixture was cooled to 50° C. and the solution was seeded with 3.125 mg/g of micronised seed (Batch “B”, Friesland Foods Domo, Netherlands). The mixture was cooled to 20° C. over 10 hours using an inverse cooling profile described by the equation T(t)=Tᵢ−(Tᵢ−T_{f})(t/t_{f})³, where T(t)=temperature at time t, tᵢ=initial temperature, T_{f}=final temperature and t_{f}=batch time. The crystallized material was then filtered using a Buchner filter and the solid washed with 30 ml of 40% acetone/water and then with 30 ml of acetone. The wet solid was slurred in 2×50 ml of acetone, filtered and air dried. The dried solid was further dried in vacuo at 40° C. overnight. The particle size distribution was measured using Sympatec HELOS particle size analysis (See Appendix 1). X50 was 25.42.

### Example 53

### Crystallization Procedure

The procedure was performed in a HEL AutoMate reactor system (Hazard Evaluation Laboratories Barnet, London). 32 g of lactose (Friesland Foods Domo, Netherlands) was dissolved in 30 ml of deionised water at 85-90° C. Sodium hydroxide solution (0.5 N, 0.3 ml) was added to the hot solution. The mixture was cooled to 50° C. and the solution was seeded with 0.75 mg/g of micronised seed (Batch “B”, Friesland Foods Domo, Netherlands). The mixture was cooled to 20° C. over 10 hours using an inverse cooling profile described by the equation T(t)=Tᵢ−(Tᵢ−T_{f})(t/t_{f})³, where T(t)=temperature at time t, tᵢ=initial temperature, T_{f}=final temperature and t_{f}=batch time. The crystallized material was then filtered using a Buchner filter and the solid washed with 30 ml of 40% acetone/water and then with 30 ml of acetone. The wet solid was slurred in 2×50 ml of acetone, filtered and air dried. The dried solid was further dried in vacuo at 40° C. overnight. The particle size distribution was measured using Sympatec HELOS particle size analysis (See Appendix 1). X50 was 37.5.

### Example 54

### Crystallization Procedure

The procedure was performed in a HEL AutoMate reactor system (Hazard Evaluation Laboratories Barnet, London). 32 g of lactose (Friesland Foods Domo, Netherlands) was dissolved in 30 ml of deionised water at 85-90° C. Sodium hydroxide solution (0.5 N, 0.3 ml) was added to the hot solution. The mixture was cooled to 50° C. and the solution was seeded with 0.1 mg/g of micronised seed (Batch “B”, Friesland Foods Domo, Netherlands). The mixture was cooled to 20° C. over 10 hours using an inverse cooling profile described by the equation T(t)=Tᵢ−(Tᵢ−T_{f})(t/t_{f})³, where T(t)=temperature at time t, tᵢ=initial temperature, T_{f}=final temperature and t_{f}=batch time. The crystallized material was then filtered using a Buchner filter and the solid washed with 30 ml of 40% acetone/water and then with 30 ml of acetone. The wet solid was slurred in 2×50 ml of acetone, filtered and air dried. The dried solid was further dried in vacuo at 40° C. overnight. The particle size distribution was measured using Sympatec HELOS particle size analysis (See Appendix 1). X50 was 74.8.

### Example 55

### Crystallization Procedure

The procedure was performed in a HEL AutoMate reactor system (Hazard Evaluation Laboratories Barnet, London). 32 g of lactose (from Friesland Foods Domo, Netherlands) was dissolved in 30 ml of deionised water at 85-90° C. Sodium hydroxide solution (0.5 N, 0.3 ml) was added to the hot solution. The mixture was cooled to 50° C. and the solution was seeded with 0.1 mg/g of micronised seed (Batch “B”, Friesland Foods Domo, Netherlands). The mixture was cooled to 20° C. over 10 hours using an inverse cooling profile described by the equation T(t)=Tᵢ−(Tᵢ−T_{f})(t/t_{f})³, where T(t)=temperature at time t, tᵢ=initial temperature, T_{f}=final temperature and t_{f}=batch time. The crystallized material was then filtered using a Buchner filter and the solid washed with 30 ml of 40% acetone/water and then with 30 ml of acetone. The wet solid was slurred in 2×50 ml of acetone, filtered and air dried. The dried solid was further dried in vacuo at 40° C. overnight. The particle size distribution was measured using Sympatec HELOS particle size analysis (See Appendix 1). X50 was 79.4.

### Example 56

### Crystallization Procedure

The procedure was performed in a HEL AutoMate reactor system (Hazard Evaluation Laboratories Barnet, London). 32 g of lactose (Friesland Foods Domo, Netherlands) was dissolved in 30 ml of deionised water at 85-90° C. Sodium hydroxide solution (0.5 N, 0.3 ml) was added to the hot solution. The mixture was cooled to 50° C. and the solution was seeded with 0.75 mg/g of micronised seed (Batch “B”, Friesland Foods Domo, Netherlands). The mixture was cooled to 20° C. over 10 hours using an inverse cooling profile described by the equation T(t)=Tᵢ−(Tᵢ−T_{f})(t/t_{f})³, where T(t)=temperature at time t, tᵢ=initial temperature, T_{f}=final temperature and t_{f}=batch time. The crystallized material was then filtered using a Buchner filter and the solid washed with 30 ml of 40% acetone/water and then with 30 ml of acetone. The wet solid was slurred in 2×50 ml of acetone, filtered and air dried. The dried solid was further dried in vacuo at 40° C. overnight. The particle size distribution was measured using Sympatec HELOS particle size analysis (See Appendix 1). X50 was 45.2.

### Example 57

### Crystallization Procedure

The procedure was performed in a HEL AutoMate reactor system (Hazard Evaluation Laboratories Barnet, London). 32 g of lactose (Friesland Foods Domo, Netherlands) was dissolved in 30 ml of deionised water at 85-90° C. Sodium hydroxide solution (0.5 N, 0.3 ml) was added to the hot solution. The mixture was cooled to 50° C. and the solution was seeded with 0.63 mg/g of directly crystallized seed (made according to copending application filed concurrently herewith entitled “Process for Manufacturing Lactose”, Ser. No. 60/821,871; X50 of 5.92 μm). The mixture was cooled to 20° C. over 10 hours using an inverse cooling profile described by the equation T(t)=Tᵢ−(Tᵢ−T_{f})(t/t_{f})³, where T(t)=temperature at time t, tᵢ=initial temperature, T_{f}=final temperature and t_{f}=batch time. The crystallized material was then filtered using a Buchner filter and the solid washed with 30 ml of 40% acetone/water and then with 30 ml of acetone. The wet solid was slurred in 2×50 ml of acetone, filtered and air dried. The dried solid was further dried in vacuo at 40° C. overnight. The particle size distribution was measured using Sympatec HELOS particle size analysis (See Appendix 1). X50 was 109.12.

### Example 58

### Crystallization Procedure

The procedure was performed in a HEL AutoMate reactor system (Hazard Evaluation Laboratories Barnet, London). 32 g of lactose (Friesland Foods Domo, Netherlands) was dissolved in 30 ml of deionised water at 85-90° C. Sodium hydroxide solution (0.5 N, 0.3 ml) was added to the hot solution. The mixture was cooled to 50° C. and the solution was seeded with 0.19 mg/g of directly crystallized seed (made according to copending application filed concurrently herewith entitled “Process for Manufacturing Lactose”, Ser. No. 60/821,871; X50 of 5.92 μm). The mixture was cooled to 20° C. over 10 hours using an inverse cooling profile described by the equation T(t)=Tᵢ−(Tᵢ−T_{f})(t/t_{f})³, where T(t)=temperature at time t, tᵢ=initial temperature, T_{f}=final temperature and t_{f}=batch time. The crystallized material was then filtered using a Buchner filter and the solid washed with 30 ml of 40% acetone/water and then with 30 ml of acetone. The wet solid was slurred in 2×50 ml of acetone, filtered and air dried. The dried solid was further dried in vacuo at 40° C. overnight. The particle size distribution was measured using Sympatec HELOS particle size analysis (See Appendix 1). X50 was 132.71.

### Example 59

### Crystallization Procedure

The procedure was performed in a HEL AutoMate reactor system (Hazard Evaluation Laboratories Barnet, London). 32 g of lactose (Friesland Foods Domo, Netherlands) was dissolved in 30 ml of deionised water at 85-90° C. Sodium hydroxide solution (0.5 N, 0.3 ml) was added to the hot solution. The mixture was cooled to 50° C. and the solution was seeded with 0.95 mg/g of directly crystallized seed (made according to copending application Ser. No. 60/821,871 entitled “Process for Manufacturing Lactose” filed concurrently herewith; X50 of 5.92 μm). The mixture was cooled to 20° C. over 10 hours using an inverse cooling profile described by the equation T(t)=Tᵢ−(Tᵢ−T_{f})(t/t_{f})³, where T(t)=temperature at time t, tᵢ=initial temperature, T_{f}=final temperature and t_{f}=batch time. The crystallized material was then filtered using a Buchner filter and the solid washed with 30 ml of 40% acetone/water and then with 30 ml of acetone. The wet solid was slurred in 2×50 ml of acetone, filtered and air dried. The dried solid was further dried in vacuo at 40° C. overnight. The particle size distribution was measured using Sympatec HELOS particle size analysis (See Appendix 1). X50 was 96.51.

### Example 60

### Crystallization Procedure

The procedure was performed in a HEL AutoMate reactor system (Hazard Evaluation Laboratories Barnet, London). 32 g of lactose (Friesland Foods Domo, Netherlands) was dissolved in 30 ml of deionised water at 85-90° C. Sodium hydroxide solution (0.5 N, 0.3 ml) was added to the hot solution. The mixture was cooled to 50° C. and the solution was seeded with 1.63 mg/g of directly crystallized seed (made according to copending application Ser. No. 60/821,871 entitled “Process for Manufacturing Lactose” filed concurrently herewith; X50 of 5.92 μm). The mixture was cooled to 20° C. over 10 hours using an inverse cooling profile described by the equation T(t)=Tᵢ−(Tᵢ−T_{f})(t/t_{f})³, where T(t)=temperature at time t, tᵢ=initial temperature, T_{f}=final temperature and t_{f}=batch time. The crystallized material was then filtered using a Buchner filter and the solid washed with 30 ml of 40% acetone/water and then with 30 ml of acetone. The wet solid was slurred in 2×50 ml of acetone, filtered and air dried. The dried solid was further dried in vacuo at 40° C. overnight. The particle size distribution was measured using Sympatec HELOS particle size analysis (See Appendix 1). X50 was 91.27.

### Example 61

### Crystallization Procedure

The procedure was performed in a HEL AutoMate reactor system (Hazard Evaluation Laboratories Barnet, London). 32 g of lactose (Friesland Foods Domo, Netherlands) was dissolved in 30 ml of deionised water at 85-90° C. Sodium hydroxide solution (0.5 N, 0.3 ml) was added to the hot solution. The mixture was cooled to 50° C. and the solution was seeded with 0.35 mg/g of directly crystallized seed (made according to copending application Ser. No. 60/821,871 entitled “Process for Manufacturing Lactose” filed concurrently herewith; X50 of 5.92 μm). The mixture was cooled to 20° C. over 10 hours using an inverse cooling profile described by the equation T(t)=Tᵢ−(Tᵢ−T_{f})(t/t_{f})³, where T(t)=temperature at time t, tᵢ=initial temperature, T_{f}=final temperature and t_{f}=batch time. The crystallized material was then filtered using a Buchner filter and the solid washed with 30 ml of 40% acetone/water and then with 30 ml of acetone. The wet solid was slurred in 2×50 ml of acetone, filtered and air dried. The dried solid was further dried in vacuo at 40° C. overnight. The particle size distribution was measured using Sympatec HELOS particle size analysis (See Appendix 1). X50 was 124.42.

### Example 62

### Crystallization Procedure

The procedure was performed in a HEL AutoMate reactor system (Hazard Evaluation Laboratories Barnet, London). 32 g of lactose (Friesland Foods Domo, Netherlands) was dissolved in 30 ml of deionised water at 85-90° C. Sodium hydroxide solution (0.5 N, 0.3 ml) was added to the hot solution. The mixture was cooled to 50° C. and the solution was seeded with 3.00 mg/g of directly crystallized seed (made according to copending application Ser. No. 60/821,871 entitled “Process for Manufacturing Lactose” filed concurrently herewith; X50 of 5.92 μm). The mixture was cooled to 20° C. over 10 hours using an inverse cooling profile described by the equation T(t)=Tᵢ−(Tᵢ−T_{f})(t/t_{f})³, where T(t)=temperature at time t, tᵢ=initial temperature, T_{f}=final temperature and t_{f}=batch time. The crystallized material was then filtered using a Buchner funnel) and the solid washed with 30 ml of 40% acetone/water and then with 30 ml of acetone. The wet solid was slurred in 2×50 ml of acetone, filtered and air dried. The dried solid was further dried in vacuo at 40° C. overnight. The particle size distribution was measured using Sympatec HELOS particle size analysis (See Appendix 1). X50 was 73.5.

### Example 63

### Crystallization Procedure

The procedure was performed in a HEL AutoMate reactor system (Hazard Evaluation Laboratories Barnet, London). 32 g of lactose (Friesland Foods Domo, Netherlands) was dissolved in 30 ml of deionised water at 85-90° C. Sodium hydroxide solution (0.5 N, 0.3 ml) was added to the hot solution. The mixture was cooled to 50° C. and the solution was seeded with 5.0 mg/g of directly crystallized seed (made according to copending application Ser. No. 60/821,871 entitled “Process for Manufacturing Lactose” filed concurrently herewith; X50 of 5.92 μm). The mixture was cooled to 20° C. over 10 hours using an inverse cooling profile described by the equation T(t)=Tᵢ−(Tᵢ−T_{f})(t/t_{f})³, where T(t)=temperature at time t, tᵢ=initial temperature, T_{f}=final temperature and t_{f}=batch time. The crystallized material was then filtered using a Buchner filter and the solid washed with 30 ml of 40% acetone/water and then with 30 ml of acetone. The wet solid was slurred in 2×50 ml of acetone, filtered and air dried. The dried solid was further dried in vacuo at 40° C. overnight. The particle size distribution was measured using Sympatec HELOS particle size analysis (See Appendix 1). X50 was 65.3.

### Example 64

### Crystallization Procedure

The procedure was performed in a HEL AutoMate reactor system (Hazard Evaluation Laboratories Barnet, London). 32 g of lactose (Friesland Foods Domo, Netherlands) was dissolved in 30 ml of deionised water at 85-90° C. Sodium hydroxide solution (0.5 N, 0.3 ml) was added to the hot solution. The mixture was cooled to 50° C. and the solution was seeded with 0.50 mg/g of directly crystallized seed (made according to copending application Ser. No. 60/821,871 entitled “Process for Manufacturing Lactose” filed concurrently herewith; X50 of 5.92 μm). The mixture was cooled to 20° C. over 10 hours using an inverse cooling profile described by the equation T(t)=Tᵢ−(Tᵢ−T_{f})(t/t_{f})³, where T(t)=temperature at time t, tᵢ=initial temperature, T_{f}=final temperature and t_{f}=batch time. The crystallized material was then filtered using a Buchner filter and the solid washed with 30 ml of 40% acetone/water and then with 30 ml of acetone. The wet solid was slurred in 2×50 ml of acetone, filtered and air dried. The dried solid was further dried in vacuo at 40° C. overnight. The particle size distribution was measured using Sympatec HELOS particle size analysis (See Appendix 1). X50 was 119.8.

### Example 65

### Crystallization Procedure

The procedure was performed in a HEL AutoMate reactor system (Hazard Evaluation Laboratories Barnet, London). 32 g of lactose (Friesland Foods Domo, Netherlands) was dissolved in 30 ml of deionised water at 85-90° C. Sodium hydroxide solution (0.5 N, 0.3 ml) was added to the hot solution. The mixture was cooled to 50° C. and the solution was seeded with 1.50 mg/g of directly crystallized seed (made according to copending application Ser. No. 60/821,871 entitled “Process for Manufacturing Lactose” filed concurrently herewith; X50 of 5.92 μm). The mixture was cooled to 20° C. over 10 hours using an inverse cooling profile described by the equation T(t)=Tᵢ−(Tᵢ−T_{f})(t/t_{f})³, where T(t)=temperature at time t, tᵢ=initial temperature, T_{f}=final temperature and t_{f}=batch time. The crystallized material was then filtered using a Buchner filter and the solid washed with 30 ml of 40% acetone/water and then with 30 ml of acetone. The wet solid was slurred in 2×50 ml of acetone, filtered and air dried. The dried solid was further dried in vacuo at 40° C. overnight. The particle size distribution was measured using Sympatec HELOS particle size analysis (See Appendix 1). X50 was 92.9.

### Example 66

### Crystallization Procedure

The procedure was carried out in a 10 litre, jacketed, controlled laboratory reactor with an attached Huber Unistat 385 HT heater chiller unit 2.6 kg of lactose (Friesland Foods Domo, Netherlands) was dissolved in 2.1 litres of deionised water. Sodium hydroxide solution (0.5 M, 24 ml) was added to the hot solution. The mixture was cooled to 50° C. and the solution was seeded with 0.31 mg/g of micronised seed (Friesland Foods Domo, Netherlands). The mixture was cooled to 20° C. over 5 hours using a linear ramp. The crystallized material was filtered in a 25 cm dia PTFE nitrogen blanketed, pan filter, pulled dry for 10 minutes and washed with 1.2 litres of 40% acetone water The cake was washed a second time with 1.2 litres of acetone and pulled dry for 10 minutes. The solid was slurried in 2.0 litres of acetone, filtered and pulled dry for 10 minutes. The solid was dried in a Salvis vacucenter, vacuum oven at 40° C. X50 was 75.71.

### Example 67

### Crystallization Procedure

The procedure was carried out in a 10 litre, jacketed, controlled laboratory reactor with an attached Huber Unistat 385 HT heater chiller unit 2.6 kg of lactose (Friesland Foods Domo, Netherlands) was dissolved in 2.1 litres of deionised water. Sodium hydroxide solution (0.5 M, 24 ml) was added to the hot solution. The mixture was cooled to 50° C. and the solution was seeded with 0.31 mg/g of micronised seed (Friesland Foods Domo, Netherlands). The mixture was cooled to 20° C. over 5 hours using a linear ramp. The crystallized material was filtered in a 25 cm dia PTFE nitrogen blanketed, pan filter, pulled dry for 10 minutes and washed with 1.2 litres of 40% acetone water The cake was washed a second time with 1.2 litres of acetone and pulled dry for 10 minutes. The solid was slurried in 2.0 litres of acetone, filtered and pulled dry for 10 minutes. The solid was dried in a Salvis vacucenter, vacuum oven at 40° C. X50 was 64.41.

### Example 68

### Crystallization Procedure

The procedure was carried out in a 10 litre, jacketed, controlled laboratory reactor with an attached Huber Unistat 385 HT heater chiller unit. 2.6 kg of lactose (Friesland Foods Domo, Netherlands) was dissolved in 2.1 litres of deionised water. Sodium hydroxide solution (0.5 M, 24 ml) was added to the hot solution. The mixture was cooled to 50° C. and the solution was seeded with 0.31 mg/g of micronised seed (Friesland Foods Domo, Netherlands). The mixture was cooled to 20° C. over 5 hours using a linear ramp. The crystallized material was filtered in a 25 cm dia PTFE nitrogen blanketed, pan filter, pulled dry for 10 minutes and washed with 1.2 litres of 40% acetone water The cake was washed a second time with 1.2 litres of acetone and pulled dry for 10 minutes. The solid was slurried in 2.0 litres of acetone, filtered and pulled dry for 10 minutes. The solid was dried in a Salvis vacucenter, vacuum oven at 40° C. X50 was 65.68.

### Example 69

### Crystallization Procedure

The procedure was carried out in a 10 litre, jacketed, controlled laboratory reactor with an attached Huber Unistat 385 HT heater chiller unit. 2.6 kg of lactose (Friesland Foods Domo, Netherlands) was dissolved in 2.1 litres of deionised water. Sodium hydroxide solution (0.5 M, 24 ml) was added to the hot solution. The mixture was cooled to 50° C. and the solution was seeded with 0.46 mg/g of micronised seed (Friesland Foods Domo, Netherlands). The mixture was cooled to 20° C. over 5 hours using a linear ramp. The crystallized material was filtered in a 25 cm dia PTFE nitrogen blanketed, pan filter, pulled dry for 10 minutes and washed with 1.2 litres of 40% acetone water The cake was washed a second time with 1.2 litres of acetone and pulled dry for 10 minutes. The solid was slurried in 2.0 litres of acetone, filtered and pulled dry for 10 minutes. The solid was dried in a Salvis vacucenter, vacuum oven at 40° C. X50 was 105.85.

### Example 70

### Crystallization Procedure

32 g of lactose (Friesland Foods Domo, Netherlands) was dissolved in 30 ml of water at 85-90° C. The pH of the solution was measured. 0.3 ml of 0.5 M sodium hydroxide solution was added to the hot solution. The mixture was cooled to 50° C. and the solution was seeded with 0.313 mg/g [per g of input material] (Friesland Foods Domo, Netherlands). The solution was then cooled to 20° C. over 10 hours using an inverse cooling profile. The material was filtered and the solid washed with 30 ml of 40% acetone/water and 2×30 ml of acetone. The cake was slurried in 2×40 ml of acetone and pulled dry for 1 hour (using a Buchner filter) and the solid unloaded and dried in vacuo (Gallenkamp vacuum oven) at 40° C. overnight. The particle size distribution was measured using Sympatec HELOS particle size analysis (See Appendix 1). X50 was 50.19.

### Example 71

### Crystallization Procedure

This procedure was performed on a 32 g scale in the HEL AutoMATE (Hazard Evaluation Laboratories Barnet, London). 32 g of lactose (Friesland Foods Domo, Netherlands) was dissolved in 30 ml of deionised water. 0.3 ml of 0.5 M sodium hydroxide solution was added to the hot solution. 0.31 mg/g Seed (Friesland Foods Domo, Netherlands) was suspended in 0.4 ml of acetone and the mixture shaken for a few seconds. The suspension was sonicated in an ultrasound bath (Branson 1510. Branson Ultrasonic Corp. Danbury, Conn., USA) for 15 seconds at ambient temperature before adding the slurry to the supersaturated solution at 50° C. The seed slurry container was rinsed into the reaction vessel with a further 0.4 ml of acetone. The seeded solution was cooled linearly over 5 hours to 20° C. The material was filtered and the solid washed with 30 ml of 40% acetone/water and 2×30 ml of acetone. The cake was slurried in 2×40 ml of acetone and pulled dry for 1 hour (using a Buchner filter) and the solid unloaded and dried in vacuo (Gallenkamp vacuum oven) at 40° C. overnight. The particle size distribution was measured using Sympatec HELOS particle size analysis (See Appendix 1) X50 was 39.63.

### Example 72

### Crystallization Procedure

This procedure was performed on a 32 g scale in the HEL AutoMATE (Hazard Evaluation Laboratories Barnet, London). 32 g of lactose (Friesland Foods Domo, Netherlands) was dissolved in 30 ml of deionised water. 0.3 ml of 0.5 M sodium hydroxide solution was added to the hot solution. 1.25 mg/g Seed (Friesland Foods Domo, Netherlands) was suspended in 0.4 ml of acetone and the mixture shaken for a few seconds. The suspension was sonicated in an ultrasound bath (Branson 1510. Branson Ultrasonic Corp. Danbury, Conn., USA) for 15 seconds at ambient temperature before adding the slurry to the supersaturated solution at 50° C. The seed slurry container was rinsed into the reaction vessel with a further 0.4 ml of acetone. The seeded solution was cooled linearly over 5 hours to 20° C. The material was filtered and the solid washed with 30 ml of 40% acetone/water and 2×30 ml of acetone. The cake was slurried in 2×40 ml of acetone and pulled dry for 1 hour (using a Buchner filter) and the solid unloaded and dried in vacuo (Gallenkamp vacuum oven) at 40° C. overnight. The particle size distribution was measured using Sympatec HELOS particle size analysis (See Appendix 1) X50 was 27.55.

### Example 73

### Crystallization Procedure

This procedure was performed on a 32 g scale in the HEL AutoMATE (Hazard Evaluation Laboratories Barnet, London). 32 g of lactose (Friesland Foods Domo, Netherlands) was dissolved in 30 ml of deionised water. 0.3 ml of 0.5 M sodium hydroxide solution was added to the hot solution. 0.156 mg/g seed (Friesland Foods Domo, Netherlands) was suspended in 0.4 ml of acetone and the mixture shaken for a few seconds. The suspension was sonicated in an ultrasound bath (Branson 1510. Branson Ultrasonic Corp. Danbury, Conn., USA) for 15 seconds at ambient temperature before adding the slurry to the supersaturated solution at 50° C. The seed slurry container was rinsed into the reaction vessel with a further 0.4 ml of acetone. The seeded solution was cooled linearly over 5 hours to 20° C. The material was filtered and the solid washed with 30 ml of 40% acetone/water and 2×30 ml of acetone. The cake was slurried in 2×40 ml of acetone and pulled dry for 1 hour (using a Buchner filter) and the solid unloaded and dried in vacuo (Gallenkamp vacuum oven) at 40° C. overnight. The particle size distribution was measured using Sympatec HELOS particle size analysis (See Appendix 1) X50 was 42.03.

### Example 74

### Crystallization Procedure

This procedure was performed on a 32 g scale in the HEL AutoMATE (Hazard Evaluation Laboratories Barnet, London). 32 g of lactose (Friesland Foods Domo, Netherlands) was dissolved in 30 ml of deionised water. 0.3 ml of 0.5 M sodium hydroxide solution was added to the hot solution. 0.625 mg/g Seed (Friesland Foods Domo, Netherlands) was suspended in 0.4 ml of acetone and the mixture shaken for a few seconds. The suspension was sonicated in an ultrasound bath (Branson 1510. Branson Ultrasonic Corp. Danbury, Conn., USA) for 15 seconds at ambient temperature before adding the slurry to the supersaturated solution at 50° C. The seed slurry container was rinsed into the reaction vessel with a further 0.4 ml of acetone. The seeded solution was cooled linearly over 5 hours to 20° C. The material was filtered and the solid washed with 30 ml of 40% acetone/water and 2×30 ml of acetone. The cake was slurried in 2×40 ml of acetone and pulled dry for 1 hour (using a Buchner filter) and the solid unloaded and dried in vacuo (Gallenkamp vacuum oven) at 40° C. overnight. The particle size distribution was measured using Sympatec HELOS particle size analysis (See Appendix 1) X50 was 29.53.

### Example 75

### Crystallization Procedure

This procedure was performed on a 32 g scale in the HEL AutoMATE (Hazard Evaluation Laboratories Barnet, London). 32 g of lactose (Friesland Foods Domo, Netherlands) was dissolved in 30 ml of deionised water. 0.3 ml of 0.5 M sodium hydroxide solution was added to the hot solution. 0.937 mg/g Seed (Friesland Foods Domo, Netherlands) was suspended in 0.4 ml of acetone and the mixture shaken for a few seconds. The suspension was sonicated in an ultrasound bath (Branson 1510. Branson Ultrasonic Corp. Danbury, Conn., USA) for 15 seconds at ambient temperature before adding the slurry to the supersaturated solution at 50° C. The seed slurry container was rinsed into the reaction vessel with a further 0.4 ml of acetone. The seeded solution was cooled linearly over 5 hours to 20° C. The material was filtered and the solid washed with 30 ml of 40% acetone/water and 2×30 ml of acetone. The cake was slurried in 2×40 ml of acetone and pulled dry for 1 hour (using a Buchner filter) and the solid unloaded and dried in vacuo (Gallenkamp vacuum oven) at 40° C. overnight. The particle size distribution was measured using Sympatec HELOS particle size analysis (See Appendix 1) X50 was 23.99.

### Example 76

### Crystallization Procedure

This procedure was performed on a 32 g scale in the HEL AutoMATE (Hazard Evaluation Laboratories Barnet, London). 32 g of lactose (Friesland Foods Domo, Netherlands) was dissolved in 30 ml of deionised water. 0.3 ml of 0.5 M sodium hydroxide solution was added to the hot solution. 1.875 mg/g Seed (from Friesland Foods Domo, Netherlands) was suspended in 0.4 ml of acetone and the mixture shaken for a few seconds. The suspension was sonicated in an ultrasound bath (Branson 1510. Branson Ultrasonic Corp. Danbury, Conn., USA) for 15 seconds at ambient temperature before adding the slurry to the supersaturated solution at 50° C. The seed slurry container was rinsed into the reaction vessel with a further 0.4 ml of acetone. The seeded solution was cooled linearly over 5 hours to 20° C. The material was filtered and the solid washed with 30 ml of 40% acetone/water and 2×30 ml of acetone. The cake was slurried in 2×40 ml of acetone and pulled dry for 1 hour (using a Buchner filter) and the solid unloaded and dried in vacuo (Gallenkamp vacuum oven) at 40° C. overnight. The particle size distribution was measured using Sympatec HELOS particle size analysis (See Appendix 1) X50 was 21.45.

### Example 77

### Crystallization Procedure

This procedure was performed in a HEL AutoMate reactor system (Hazard Evaluation Laboratories Barnet, London). 32 g of lactose (Friesland Foods Domo, Netherlands) was dissolved in 30 ml of deionised water at 85-90° C. Sodium hydroxide solution (0.5 N, 0.3 ml) was added to the hot solution. The mixture was cooled to 50° C. and the solution was seeded with a suspension of 0.14 mg/g of micronised seed (Batch 403003, Friesland Foods Domo, Netherlands) in 1 ml of acetone; the micronised seed suspension was shaken before it was added to the supersaturated lactose solution. The mixture was cooled to 20° C. over 10 hours using an inverse cooling profile described by the equation T(t)=Tᵢ−(Tᵢ−T_{f})(t/t_{f})³, where T(t)=temperature at time t, tᵢ=initial temperature, T_{f}=final temperature and t_{f}=batch time. The crystallized material was then filtered using a Buchner filter and the solid washed with 30 ml of 40% acetone/water and then with 30 ml of acetone. The wet solid was slurred in 2×50 ml of acetone, filtered and air dried. The dried solid was further dried in vacuo at 40° C. overnight.

### Example 78

### Crystallization Procedure

This procedure was performed in a HEL AutoMate reactor system (Hazard Evaluation Laboratories Barnet, London). 32 g of lactose (Friesland Foods Domo, Netherlands) was dissolved in 30 ml of deionised water at 85-90° C. Sodium hydroxide solution (0.5 N, 0.3 ml) was added to the hot solution. The mixture was cooled to 50° C. and the solution was seeded with a suspension of 0.36 mg/g of micronised seed (Batch 403003, Friesland Foods Domo, Netherlands) in 1 ml of acetone; the micronised seed suspension was shaken before it was added to the supersaturated lactose solution. The mixture was cooled to 20° C. over 10 hours using an inverse cooling profile described by the equation T(t)=Tᵢ−(Tᵢ−T_{f})(t/t_{f})³, where T(t)=temperature at time t, tᵢ=initial temperature, T_{f}=final temperature and t_{f}=batch time. The crystallized material was then filtered using a Buchner filter and the solid washed with 30 ml of 40% acetone/water and then with 30 ml of acetone. The wet solid was slurred in 2×50 ml of acetone, filtered and air dried. The dried solid was further dried in vacuo at 40° C. overnight.

### Example 79

### Crystallization Procedure

This procedure was performed in a HEL AutoMate reactor system (Hazard Evaluation Laboratories Barnet, London). 32 g of lactose (Friesland Foods Domo, Netherlands) was dissolved in 30 ml of deionised water at 85-90° C. Sodium hydroxide solution (0.5 N, 0.3 ml) was added to the hot solution. The mixture was cooled to 50° C. and the solution was seeded with a suspension of 0.74 mg/g of micronised seed (Batch 403003, Friesland Foods Domo, Netherlands) in 1 ml of acetone; the micronised seed suspension was shaken before it was added to the supersaturated lactose solution. The mixture was cooled to 20° C. over 10 hours using an inverse cooling profile described by the equation T(t)=Tᵢ−(Tᵢ−T_{f})(t/t_{f})³, where T(t)=temperature at time t, tᵢ=initial temperature, T_{f}=final temperature and t_{f}=batch time. The crystallized material was then filtered using a Buchner filter and the solid washed with 30 ml of 40% acetone/water and then with 30 ml of acetone. The wet solid was slurred in 2×50 ml of acetone, filtered and air dried. The dried solid was further dried in vacuo at 40° C. overnight.

### Example 80

### Crystallization Procedure

This procedure was performed in a HEL AutoMate reactor system (Hazard Evaluation Laboratories Barnet, London). 32 g of lactose (Friesland Foods Domo, Netherlands) was dissolved in 30 ml of deionised water at 85-90° C. Sodium hydroxide solution (0.5 N, 0.3 ml) was added to the hot solution. The mixture was cooled to 50° C. and the solution was seeded with a suspension of 1.49 mg/g of micronised seed (Batch 403003, Friesland Foods Domo, Netherlands) in 1 ml of acetone; the micronised seed suspension was shaken before it was added to the supersaturated lactose solution. The mixture was cooled to 20° C. over 10 hours using an inverse cooling profile described by the equation T(t)=Tᵢ−(Tᵢ−T_{f})(t/t_{f})³, where T(t)=temperature at time t, tᵢ=initial temperature, T_{f}=final temperature and t_{f}=batch time. The crystallized material was then filtered using a Buchner filter and the solid washed with 30 ml of 40% acetone/water and then with 30 ml of acetone. The wet solid was slurred in 2×50 ml of acetone, filtered and air dried. The dried solid was further dried in vacuo at 40° C. overnight.

### Example 81

### Fine Particle Fraction (FPF) Evaluation of Formulations Using Crystallized Lactose

Various formulations of salmeterol xinafoate (SX) (50 μm) and fluticasone propionate (FP) (25 μm), per 12.5 mg concentration, were tested for FPF. The results are set forth in Table xx.

Descriptions of materials are as follows:

Coarse grade DCL—Manufactured in accordance with the teachings set forth hereinabove and harvested using a filter drier. X50 46 μm (Sympatec RODOS with 1.5 bar dispersion pressure)

Coarse grade DCL (Boltz drier)—dried using a Boltz drier in which the material is agitated as it dries. X50 47 μm. By SEM this material appeared to have a rougher surface than the filter dried material.

Fine Grade DCL—manufactured such that these specifications were acheived: X50 6.7 μm, and additional batch X50 8.3 μm.

The runs set forth in the tables below were intended to have various fines content. Sympatec RODOS dry powder dispersion was used to determine the content of fine lactose <15 μm. The FPF was determined using an Andersen cascade impactor at 60/min with an aerosolisation device. The blends were mixed using a small scale, high shear blender (QMM Micrometer, 2.5 L bowl, QMM Micromixer, Donsmark Process. Technology, Denmark) with a 500 g batch size. The FP was added to the lactose and blended at 600 rpm for 5 min and thereafter the SX was added and blended for 9 min at 600 rpm. The active ingredients were added to the lactose.

Runs containing course milled and classified lactose and fine milled and classified are also set forth (classified to produce a coarse grade with X50 90 μm and fine grade with X50 22 μm), Friesland Foods Domo, Netherlands.

FPF results are set forth in Tables 3 and 4.

TABLE 3

| Coarse | Fine | | | |
|---|---|---|---|---|
| DCL | DCL | % w/w < 15 μm | FP FPF (% of | SX FPF (% of |
| % w/w | % w/w | by Sympatec | emitted dose) | emitted dose) |
| 95 | 5 | 9.95 | 16.3 | 12.8 |
| 92.5 | 7.5 | 12.9 | 17.8 | 14.8 |
| 90 | 10 | 15.8 | 19.2 | 16.6 |
| 85 | 15 | 21.21 | 19.7 | 17.8 |
| 80 | 20 | 26.4 | 17.9 | 18.1 |
| 75 | 25 | 31.6 | 16.0 | 19.7 |
| 90 (Boltz | 10 | 16.4 | 23.9 | 21.8 |
| dried) | | | | |
| 90 | 10 (X50 | 14.7 | 14.5 | 12.1 |
| | 8.3 μm) | | | |

TABLE 4

| | | | FPF (% of | FPF (% of |
|---|---|---|---|---|
| Coarse milled | Fine milled | % w/w < | emitted | emitted |
| and classified | and classified | 15 μm | dose) | dose) |
| 95 | 5 | 6.9 | 23.8 | 21.4 |
| 85 | 15 | 13.0 | 33.2 | 30.2 |
| 75 | 25 | 18.0 | 36.4 | 37.0 |

### Example 82

### Fine Particle Fraction (FPF) Evaluation of Formulations Using Crystallized Lactose

Micronized 4-{(1R)-2-[(6-{2-[(2,6-dichlorobenzyl)oxy]ethoxy}hexyl)amino]-1-hydroxyethyl}-2-(hydroxymethyl)phenol was used as the active agent at 12.5 μg/mg (not corrected for salt) for the formulations containing lactose from Friesland Foods Domo (“conv”) and 12.5 μg/9.76 mg for the direct crystallized lactose (“DCL”) formulations according to the invention. The differing blend concentration was undertaken as DCL has a 25% lower bulk density than FFD lactose thus allowing all formulations to be targeted at 12.5 μg (salt) per blister.

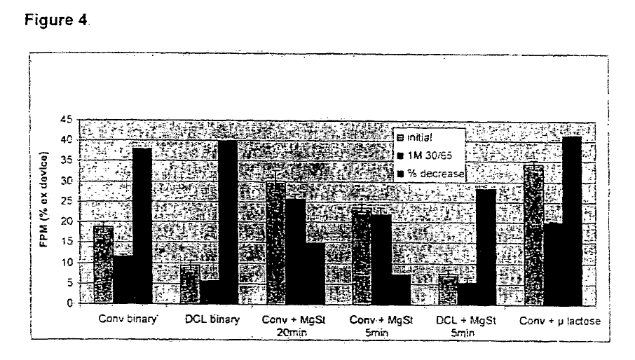

The Input Materials Used were:
Milled and classified lactose, Friesland Foods Domo (“Conv”). Coarse grade Batch lot “10”, Fine grade lot “11”
Directly crystallised lactose (“DCL”). Coarse grade Batch “12”, Fine Grade Batch “13”
Micronised lactose. Microfine Batch “14”, Friesland Foods, Netherlands
Magnesium Stearate. Liga Magnesium Stearate MF-2-V Vegetable (“MgSt”)
The composition of the formulations is summarized in Table 5 below. The FPF was determined with the Andersen cascade impactor at 60 l/min using a Diskus device.
The primary pack was compromised by puncturing with a 0.8 mm diameter pin and the blister strips were stored at 30° C./65% RH for a month. The FPF was remeasured. The FPF data are presented in FIG. 4.

TABLE 5

| | | | | | Mag. | |
|---|---|---|---|---|---|---|
| | Coarse grade | | Fine grade | | Stearate | |
| Batch no | Type | % w/w | Type | % w/w | (% w/w) | Blending procedure |
| 20 | Conv | 95 | Conv | 5 | 0 | All materials blended |
| | | | | | | at 500 rpm for 2 mins |
| 30 | DCL | 92.5 | DCL | 7.5 | 0 | All materials blended |
| | | | | | | for 5 mins at 500 rpm |
| 40 | Conv | 95 | Conv | 5 | 0.25 | All materials blended |
| | | | | | | together at 500 rpm |
| | | | | | | for 5 mins |
| 50 | Conv | 95 | conv | 5 | 0.25 | Lactose and MgSt |
| | | | | | | blended together at |
| | | | | | | 750 rpm for 10 mins |
| | | | | | | then drug added for |
| | | | | | | 10 mins at 750 rpm |
| 60 | DCL | 92.5 | DCL | 7.5 | 0.25 | All materials blended |
| | | | | | | together for 5 mins at |
| | | | | | | 500 rpm |
| 70 | conv | 94 | Micronized | 6 | 0 | All materials blended |
| | | | | | | together for 2 mins at |
| | | | | | | 500 rpm |

The invention has be illustrated by the embodiments in the specification, including those in the drawings and examples. It should be understood that these embodiments do not limit the scope of the invention as defined by the claims.

What is claimed is:

1. A process for producing lactose particles, said process comprising:
combining a predetermined quantity of lactose seed particles to a first aqueous solution comprising a plurality of lactose particles to form a second solution, wherein the predetermined quantity of lactose seed particles is present in a well dispersed suspension and the first aqueous solution is saturated with said plurality of lactose particles;
subjecting said second solution to conditions sufficient to induce crystallization of the lactose seed particles to form a second plurality of lactose particles having a median particle size of about 25 microns to about 100 microns.

2. The process according to claim 1, wherein the pH of the second solution ranges from about pH 4 to about pH 9.

3. The process according to claim 1, wherein the well dispersed suspension of lactose particles comprises a water miscible nonsolvent.

4. The process according to claim 3, wherein the water miscible nonsolvent is selected from the group consisting of acetone, ethanol, methanol, isopropyl alcohol, tetrahydrofuran, methyl ethyl ketone, and mixtures thereof.

5. The process according to claim 4, wherein the water miscible nonsolvent is acetone.

6. The process according to claim 1, wherein the temperature of said first aqueous solution ranges from about 60° C. to about 40° C.

7. The process according to claim 1, wherein the second plurality of lactose particles having a median particle size of about 50 microns comprises α-lactose monohydrate.

8. The process according to claim 1, wherein the predetermined quantity of lactose seed particles is determined according to the equation:
m_{Seed}=d³_{Seed}/d³_{product}×m_{product}×c
wherein:
m_{Seed} represents the mass of the lactose Seed particles;
d_{Seed} represents the X50 of the lactose Seed particles;
d_{product} represents the X50 of the plurality of lactose particles formed by said process; and
m_{product} represents the mass of the plurality of lactose particles formed by said process
c represents a Seed constant determined by experimental measurement.

9. The process according to claim 1, wherein prior to said step of adding a predetermined quantity of lactose seed particles to a first aqueous solution, said process further comprises:
providing an aqueous solution having a first pH;
adjusting the first pH of the aqueous solution by adding a solution comprising an inorganic base such that the aqueous solution has a second pH; and
subjecting the aqueous solution to conditions sufficient to cool the aqueous solution.

10. The process according to claim 9, wherein the first pH of the aqueous solution ranges from about 3 to about 7.

11. The process according to claim 9, wherein the second pH of the aqueous solution ranges from about 5 to about 9.

12. The process according to claim 9, wherein the inorganic base is selected from the group consisting of NaOH, KOH, LiOH, and NaHCO₃.

13. The process according to claim 9, wherein the inorganic base is NaOH.

14. The process according to claim 9, wherein said step of subjecting the aqueous solution to conditions sufficient to cool the aqueous solution comprises cooling the aqueous solution to a temperature ranging from about 60° C. to about 40° C.

15. The process according to claim 14, wherein the temperature is about 50° C.

16. The process according to claim 1, wherein subsequent to said step of subjecting said second solution to conditions sufficient to induce crystallization of the lactose seed particles to form a second plurality of lactose particles, said process further comprises:
subjecting said second solution having a plurality of lactose particles with an average particle size of about 50 microns to conditions sufficient to cool said second solution;
adding a solution comprising an anti-solvent to the cooled second solution to isolate the plurality of lactose particles with an average particle size of about 50 microns.

17. The process according to claim 16, wherein said step of subjecting said second solution having a plurality of lactose particles with an average particle size of about 50 microns to conditions sufficient to cool said second solution comprises cooling said second solution to a temperature ranging from about 30° C. to about 0° C.

18. The process according to claim 16, wherein the anti-solvent is selected from the group consisting of acetone, methanol, ethanol, iso-propanol, n-propanol, and tetrahydrofuran and mixtures thereof.

19. The process according to claim 16, further comprising:
processing the isolated crystallized lactose particles such that the processed crystallized lactose particles are suitable for use in a pharmaceutical formulation; and
combining the processed crystallized lactose particles with at least one medicament to form a pharmaceutical formulation.

20. The process according to claim 19, where said step of
processing the isolated crystallized lactose particles comprises:
filtering said isolated crystallized lactose particles to form filtered crystallized lactose particles;
washing said filtered crystallized lactose particles; and
drying said filtered crystallized lactose particles to form the processed crystallized lactose particles.

21. The process according to claim 20, further comprising combining the resulting crystallized lactose particles with lactose particles having a median size of about 4 μm to about 20 μm.

22. The process according to claim 19, wherein the pharmaceutical formulation is a dry powder pharmaceutical formulation suitable for inhalation.

23. The process according to claim 19, wherein said at least one medicament is selected from the group consisting of analgesics, anginal preparations, antiinfectives, antihistamines, anti-inflammatories, antitussives, bronchodilators, diuretics, anticholinergics, hormones, xanthines, therapeutic proteins and peptides, salts thereof, esters thereof, solvates thereof, and combinations thereof.

24. The process according to claim 19, wherein the at least one medicament comprises at least one beta agonist.

25. The process according to claim 24, wherein the at least one beta agonist is selected from the group consisting of salbutamol, terbutaline, salmeterol, bitolterol, formoterol, 3-(4-{[6-({(2R)-2-hydroxy-2-[4-hydroxy-3-(hydroxymethyl)phenyl]ethyl}amino)hexyl]oxy}butyl)benzenesulfonamide, 3-(3-{[7-({(2R)-2-hydroxy-2-[4-hydroxy-3-(hydroxymethyl)phenyl]ethyl}amino)heptyl]oxy}propyl)benzenesulfonamide, 4-{(1R)-2-[(6-{2-[(2,6-dichlorobenzyl)oxy]ethoxy}hexyl)amino]-1-hydroxyethyl}-2-(hydroxymethyl)phenol, 2-hydroxy-5-((1R)-1-hydroxy-2-{[2-(4-{[(2R)-2-hydroxy-2-phenylethyl]amino}phenyl)ethyl]amino}ethyl)phenylformamide, 8-hydroxy-5-{(1R)-1-hydroxy-2-[(2-{4-[(6-methoxy-1,1′-biphenyl-3-yl)amino]phenyl}ethyl)amino]ethyl}quinolin-2(1H)-one, esters thereof, solvates thereof, salts thereof, and combinations thereof.

26. The process according to claim 24, wherein the at least one beta agonist comprises salmeterol xinafoate.

27. The process according to claim 24, wherein the at least one beta agonist comprises salbutamol sulphate.

28. The process according to claim 19, wherein the at least one medicament comprises at least one anti-inflammatory steroid.

29. The process according to claim 28, wherein the at least one anti-inflammatory steroid is selected from the group consisting of mometasone, beclomethasone, budesonide, fluticasone, dexamethasone, flunisolide, triamcinolone, (6α,11β,16α,17α)-6,9-difluoro-17-{[(fluoromethyl)thio]carbonyl}-11-hydroxy-16-methyl-3-oxoandrosta-1,4-dien-17-yl 2-furoate, (6α,11β,16α,17α)-6,9-difluoro-17-{[(fluoromethyl)thio]carbonyl}-11-hydroxy-16-methyl-3-oxoandrosta-1,4-dien-17-yl 4-methyl-1,3-thiazole-5-carboxylate, esters thereof, solvates thereof, salts thereof, and combinations thereof.

30. The process according to claim 28, wherein the at least one anti-inflammatory steroid comprises fluticasone propionate.

31. The process according to claim 19, wherein the at least one medicament comprises at least one beta agonist and at least one anti-inflammatory steroid.

32. The process according to claim 28, wherein the at least one beta-agonist comprises salmeterol xinafoate and the at least one anti-inflammatory steroid comprises fluticasone propionate.

33. The process according to claim 19, wherein the at least one medicament is selected from the group consisting of beclomethasone, fluticasone, flunisolide, budesonide, rofleponide, mometasone, triamcinolone, noscapine, albuterol, salmeterol, ephedrine, adrenaline, fenoterol, formoterol, isoprenaline, metaproterenol, terbutaline, tiotropium, ipatropium, phenylephrine, phenylpropanolamine, pirbuterol, reproterol, rimiterol, isoetharine, tulobuterol, (−)-4-amino-3,5-dichloro-α-[[[6-[2-(2-pyridinyl)ethoxy]hexyl]methyl]benzenemethanol, esters thereof, solvates thereof, salts thereof, and combinations thereof.

34. The process according to claim 19, wherein the at least one medicament is selected from the group consisting of albuterol sulphate, salmeterol xinafoate, fluticasone propionate, beclomethasone dipropionate, and combinations thereof.

35. The process according to claim 19, wherein said pharmaceutical formulation further comprises at least one additional excipient.
